(19) United States
(12) Patent Application Publication
(10) Pub. No.: US 2021/0236516 A1
(43) Pub. Date: Aug. 5, 2021

(54) AUTOPHAGY ACTIVATORS FOR TREATING OR PREVENTING SKIN INJURY

(71) Applicant: Case Western Reserve University, Cleveland, OH (US)

(72) Inventor: Kurt Q. Lu, Cleveland, OH (US)

(21) Appl. No.: 16/811,123

(22) Filed: Mar. 6, 2020

Related U.S. Application Data
(63) Continuation of application No. 16/099,726, filed on Nov. 8, 2018, now abandoned.
(60) Provisional application No. 62/336,159, filed on May 13, 2016.
(60) Provisional application No. 62/351,051, filed on Jun. 16, 2016.
(60) Provisional application No. 62/442,840, filed on Jan. 5, 2017.
(60) Provisional application No. 62/447,173, filed on Jan. 17, 2017.
(60) Provisional application No. 62/492,025, filed on Apr. 28, 2017.

(51) Int. Cl. A61K 31/593; A61K 31/00; A61K 31/592; A61P 17/02; A61P 29/00; A61K 9/00

(52) U.S. Cl. CPC A61K 31/593; A61K 31/00; A61K 9/0053; A61P 17/02; A61P 29/00; A61K 31/592

(57) ABSTRACT

A method of treating or preventing skin damage in a subject in need thereof is described that includes administering to the subject a therapeutically effective amount of an autophagy activator, such as vitamin D. The method can include administering doses of the autophagy activator that are substantially higher than those typically used.

### CONTINUING APPLICATION DATA

This application claims the benefit of U.S. Provisional Application Ser. No. 62/336,159, filed May 13, 2016, U.S. Provisional Application Ser. No. 62/351,051, filed Jun. 16, 2016, U.S. Provisional Application Ser. No. 62/442,840, filed Jan. 5, 2017, U.S. Provisional Application Ser. No. 62/447,173, filed Jan. 17, 2017, and U.S. Provisional Application Ser. No. 62/492,025, filed Apr. 28, 2017, all of which are incorporated by reference herein.

### GOVERNMENT FUNDING

This invention was made with government support under grant number P30-AR039750 awarded by the National Institute of Arthritis, Musculoskeletal and Skin Diseases (NIAMS) and grant number U01-AR064144 awarded by the National Institutes of Health (NIH). The government has certain rights in this invention.

### BACKGROUND

Vitamin D is a ubiquitous fat-soluble hormone important in calcium homeostasis and bone metabolism. The majority of vitamin D arises from de novo synthesis in the skin triggered by ultraviolet radiation (UVR), with smaller contributions from dietary sources. The Institute of Medicine (IOM) of the National Academy of Sciences has concluded that an Adequate Intake (AI) of Vitamin D for a healthy individual ranges from 200 to 600 IU per day, depending on the individual's age and sex. Currently available oral Vitamin D supplements typically contain 400 IU to 5,000 IU of Vitamin D₃ or 50,000 IU of Vitamin D₂ and are formulated for quick or immediate release in the gastrointestinal tract. High doses of Vitamin D supplements are avoided because they produce surges or spikes in blood and intracellular 25-hydroxyvitamin D levels, thereby promoting excessive extrarenal production of Vitamin D hormones, and leading to local aberrations in calcium and phosphorus homeostasis and increased risk of hypercalciuria, hypercalcemia and hyperphosphatemia.

While considerable attention has been placed on vitamin D deficiency and optimizing supplementation strategies, appreciation for the diverse biological effects and long-term outcomes of vitamin D is underappreciated. Investigators have demonstrated that vitamin D plays a role in modulation of immune responses, inflammatory disease, cardiovascular health, and carcinogenesis. Giovannucci et al., J. Natl. Cancer Institute 98(7):451-9 (2006); Martins et al. Archives of internal medicine 167(11):1159-65 (2007); Rosen, C J, N Engl J Med. 364(3):248-54 (2011); Sanders et al., JAMA, 303(18):1815-22 (2010); Wobke et al., Frontiers in physiology 5:244 (2014).

Keratinocytes and macrophages produce active vitamin D within the skin. Baeke et al., Current opinion in pharmacology, 10(4):482-96 (2010); Bikle D D., Mol Cell Endocrinol., 347(1-2):80-9 (2011). Vitamin D has pleiotropic effects on the immune system, including the enhancement of anti-microbial responses, induction of autophagy, and suppression of pro-inflammatory mediators, including tumor necrosis factor-α (TNF-α). Di Rosa et al., Cellular immunology, 280(1):36-43 (2012); Liu et al., Science, 311(5768):1770-3 (2006); Zhang et al., J. of Immunol., 188(5):2127-35 (2012). In addition, it was recently demonstrated that intervention with a single dose of vitamin D is capable of rapidly attenuating an inflammatory response in a mouse model of chemical induced skin injury through inhibition of inducible nitric oxide synthase (iNOS) and TNF-α by activated macrophages. Au et al., J Invest Dermatol. 135(12):2971-81 (2015). However, there is a lack of evidence demonstrating that intervention with vitamin D is capable of resolving acute inflammation in target tissues and organs in humans.

### SUMMARY OF THE INVENTION

The diverse immunomodulatory effects of vitamin D are increasingly being recognized. However, the ability of vitamin D to modulate acute inflammation in vivo has not been established in humans. In a double-blinded, placebo-controlled interventional trial, twenty healthy adults were randomized to receive either placebo or a high dose of vitamin D3 (cholecalciferol) one hour after experimental sunburn induced by an erythemogenic dose of ultraviolet radiation. Compared to placebo, participants receiving vitamin D3 (200,000 IU) demonstrated reduced expression of pro-inflammatory mediators TNF-α and iNOS in skin biopsy specimens 48 hours after experimental sunburn. A blinded, unsupervised hierarchical clustering of participants based on global gene expression profiles revealed that participants with significantly higher serum vitamin D3 levels after treatment demonstrated increased skin expression of the anti-inflammatory mediator arginase-1, and a sustained reduction in skin redness, correlating with significant expression of genes related to skin barrier repair. In contrast, participants with lower serum vitamin D3 levels had significant expression of pro-inflammatory genes.

A pilot study of 4 healthy subjects exposed to topical nitrogen mustard (Valchlor™ 0.016% gel) also revealed that the 2 subjects treated with 200,000 I.U. oral vitamin D3 intervention had demonstrated 50% reduction in skin vesication on histology. Placebo subjects had increasing skin erythema in contrast to a reduction in the vitamin D3 subjects (+0.35 vs. −0.49, respectively, slope by linear regression). qRT-PCR of the skin biopsy showed 66% reduction in TNF-α; 112% reduction in IL-6, and 80% reduction in COX-2. There was also demonstrable reduction in skin symptoms of warmth and irritation by patient self-reported outcomes. There was a trend in decreasing thickness. No iNOS was observed in the skin. Together the data may have broad implications for the immunotherapeutic properties of vitamin D in skin homeostasis, and implicate arginase-1 up regulation as a previously unreported mechanism by which vitamin D exerts anti-inflammatory effects in humans.

Cutaneous inflammation from UV exposure causes epidermal damage and cellular infiltration to exacerbate tissue destruction. The anti-inflammatory effects of vitamin D, 25(OH)D (VitD) prompted us to explore the mechanism underlying VitD protection in UV irradiated mice. A single administration of VitD ip prevented excessive cutaneous damage with sustained inhibition of TNF-α and MMP9 and accelerated skin recovery. Compared to UV alone, VitD protection was associated with enhanced autophagy (LC3 expression), an intracellular degradative process that modulates inflammation. Analyses of CD45+ myeloid cells infiltrating the skin revealed 2 distinct macrophage populations—LC3loLy6C+ M1cells expanded by UV inflammation compared to LC3hiCD206+ M2 cells that emerged following VitD treatment. Blockade of autophagy with inhibitor 3-MA exacerbated UV-induced inflammation and could not be reversed by treatment with vitD implicating a role for autophagy in vitD conferred protection. 3-MA exaggerated UV-induced epidermal apoptosis and depleted functional M2 macrophages to diminish M2:M1 ratio with worse skin damage. Consistent with our findings in UV-irradiated mice, examination of UV-exposed human skin revealed that VitD protection was similarly associated with macrophage-specific enhanced autophagy. Our findings identify an essential connection for autophagy and inflammation in VitD mediated skin protection from UV damage.

### BRIEF DESCRIPTION OF THE FIGURES

FIGS. 1A and 1B provide charts showing Study Design and Baseline Characteristics. Panel A depicts the enrollment, allocation, follow-up, and analysis of participants. Panel B depicts a schematic of the parallel phase study design. Skin erythema and thickness were measured 24 hr, 48 hr, 72 hr, and 1 week after experimental sunburn induced by an erythemogenic dose of simulated solar radiation. Participants returned two weeks after conclusion of the control phase of the study for the investigative phase, and were subsequently randomized to receive the study drug as a post-exposure treatment 1 hr after experimental sunburn on the contralateral arm. Punch biopsies for tissue TNF-α, iNOS, and microarray analyses were obtained 48 hr after experimental sunburn in both phases of the study.

FIGS. 2A-2D provide graphs and images showing the Primary Outcomes of Randomized Treatment Groups. Panel A shows representative clinical images of irradiation sites of participants in each treatment group in the control and investigative phases of the study. Panel B shows representative hematoxylin and eosin stained histological images obtained from punch biopsies from participants in each treatment group 48 hr after irradiation with 3MED. Panel C presents the difference in TNF-α and iNOS mRNA expression obtained from punch biopsies between the investigative and control phases of the study [(RNA48 hr)invest/(RNA48 hr)control]. Bars represent the mean, and error bars represent the standard error of the mean for the placebo (n=4), 50,000 IU D3 (n=5), 100,000 IU D3 (n=4), and 200,000 IU D3 (n=5) groups. Two participants were excluded from the placebo analysis given poor RNA sample quality. Panel D presents a heat map depicting global gene expression averages for each treatment group, with dendrogram depicting the unbiased hierarchical clustering of treatment groups based on similarities in gene expression profiles. Red indicates increased gene expression and green indicates decreased gene expression, correlating to a row-wise z-score. Statistical comparisons are made between vitamin D3 treatment groups and the placebo group. Abbreviations: n.s., non-significant. Scale bar 100 μm.

FIGS. 3A-3C provide graphs and images showing Unsupervised Clustering of Participants Based on Gene Expression Profiles. Panel A presents a heat map depicting global gene expression profiles for individual participants, with the resulting dendrogram depicting the unsupervised hierarchical clustering of participants based only on similarities in gene expression. Red indicates increased gene expression and green indicates decreased gene expression, correlating to a row-wise z-score. Two unique clusters emerged from this unbiased and blinded analysis. Cluster 1 was characterized by down regulation of arginase-1, and up regulation of genes involved in skin inflammation. Cluster 2 was characterized by up regulation of arginase-1 and genes involved in skin barrier repair. Panel B presents the fold change difference in skin arginase-1 mRNA expression from punch biopsies obtained in the investigative and control phases of the study [(RNA₄₈ₕᵣ)ᵢₙᵥₑₛₜ/(RNA₄₈ₕᵣ)_{control}]. Data points represent arginase-1 fold change differences for individual participants at each time point. Horizontal lines represent the mean for cluster 1 (n=11) and cluster 2 (n=7). Panel C presents immunofluorescently stained sections from a representative participant receiving 200,000 IU D3 before and after vitamin D3 intervention. Nuclear DNA is depicted in blue (DAPI), arginase-1 protein expression is depicted in red, and CD163, a marker of macrophages, is depicted in green. Arginase-1 protein levels are increased primarily within CD163+ macrophages after vitamin D3 intervention. White scale bar represents 20 micrometers. Abbreviations: FC, fold change.

FIGS. 4A-4C provide graphs showing Serum Vitamin D3 and Skin Erythema Following Experimental Sunburn in Cluster 1 and Cluster 2. Panel A presents the serum 25(OH)D₃ levels over time after study drug administration for cluster 1 (n=11) and cluster 2 (n=7). Panel B presents the serum 25(OH)D₃ levels over time after study drug administration for cluster 1 (n=7) and cluster 2 (n=7), excluding the participants from cluster 1 who received placebo (n=4). Error bars represent the standard error of the mean for each group at each time point. Panel c presents the change in erythema over time after experimental sunburn with 2MED for each cluster. Data points represent erythema values for individual participants at each time point. Horizontal lines represent the mean for cluster 1 (n=11) and cluster 2 (n=7) at each time point. Statistical comparisons are between cluster 1 and cluster 2 at each time point. * p<0.05; **p<0.01.

FIGS. 5A and 5B provide a graphic representation of the Effect of Oral Vitamin D3 Intervention on Skin Inflammation. As depicted in panel A, levels of vitamin D3 are at baseline levels in the absence of high dose oral vitamin D3 intervention. In this context, exposure to erythemogenic doses of UVR results in sunburn and the release of pro-inflammatory cytokines and chemokines in the skin, including TNF-α and iNOS, which further propagate tissue inflammation. Increased skin redness and thickness are mediated by vasodilation, an influx of inflammatory cells, and vascular congestion within the skin. The gene expression profile of skin at this time is characterized by increased expression of various pro-inflammatory genes. As depicted in panel B, levels of vitamin D3 rapidly rise within the serum after high dose oral vitamin D3 intervention. Arginase-1 is up regulated within the skin, and production of the pro-inflammatory mediators TNF-α and iNOS are attenuated after sunburn. Reduced skin erythema and thickness are observed clinically. The gene expression profile of skin at this time is characterized by increased expression of skin barrier genes, which help to repair the epidermal barrier and attenuate the inflammatory insult.

FIGS. 6A and 6B provide information regarding serum Vitamin D3 metabolites. Panel A presents the serum concentration of 25(OH)D₃ (ng/mL) for each participant at baseline, as well as 24 hr, 48 hr, 72 hr, and 1 week after administration of the study drug in the investigative phase of the study. Linear regression best-fit lines are presented for each participant. Panel B presents the change in serum concentration for the vitamin D3 metabolites 25(OH)D₃ (ng/mL), 1,25(OH)₂D3 (pg/mL), and 24,25(OH)₂D3 (ng/mL) at each time point after administration of the study drug in the investigative phase of the study. The difference in serum concentration between each time point and baseline are presented. Statistical comparisons are between vitamin D3 treatment groups and the placebo group.

FIG. 7 provides graphs showing serum calcium levels. The serum calcium concentration (mg/dL) is presented for each participant at baseline, as well as 24 hr, 48 hr, 72 hr, and 1 week after administration of the study drug in the investigative phase of the study. Linear regression best-fit lines are presented for each participant. The solid red line indicates the upper limit of the reference range for total serum calcium (10.7 mg/dL).

FIGS. 8A and 8B provide graphs showing the dose-dependent relationship between MED and non-invasive clinical outcomes. Panel A presents the difference in skin redness between irradiated and non-irradiated skin (a*_{irrad}−a*_{non-irrad}) 24 hr and 48 hr after exposure to 1MED, 2MED, and 3MED. Panel B presents the difference in skin thickness between irradiated and non-irradiated skin (th_{irrad}−th_{non-irrad}) 72 hr and 1 week after exposure to 1MED, 2MED, and 3MED. Bars represent the mean, and error bars represent the standard error of the mean for all participants in each MED group (n=20). Statistical comparisons are between the 2MED or 3MED groups and the 1MED group at each time point. * p<0.05; **p<0.005; n.s., non-significant.

FIG. 9 provides a schematic representation of a vitamin D3 clinical study with topical Valchlor.

FIG. 10 provides images of subjects after exposure to topical Valchlor™ at 1 week. Biopsy sites are delineated as well as repeat sites of Valchlor™ exposure.

FIG. 11 provides images of the histology of skin biopsies in study subjects exposed to topical Valchlor™. Biopsies are obtained 72 hours after exposure to Valchlor™ for each arm. Vesicle formation in study subject GN are demarcated with red arrows.

FIG. 12 provides a graph showing that oral vitamin D3 results in reduction of 3 key pro-inflammatory factors in NM exposed skin. The data represents the average qRT-PCR values of 2 subjects in placebo (black) and 2 in the vitamin D3 intervention group (green).

FIGS. 13A and 13B provide graphs showing patient self-reported skin symptoms using Pro-Diary™ wristwatch. The data presented shows the average difference of values comparing arm 2 and corresponding days in arm 1. Individual recorded values (arm 2-arm 1) are also plotted for each subject.

FIG. 14 provides graphs showing Patient self-reported skin symptoms using Pro-Diary™ wristwatch. The data presented shows the average difference of values comparing arm 2 and corresponding days in arm 1. A positive linear regression value indicates association with increased skin sensation. A negative linear regression value indicates decreasing skin sensation. Individual recorded values (arm 2-arm 1) are also plotted for each subject.

FIGS. 15A & 15B provide graphs and images showing VitD protects from UV-mediated skin inflammation. Dorsal back of C57BL/6 mice were subject to 100 mJ/m² UV radiation followed by 5 ng VitD 1 hour after exposure. Exposed skin tissue was excised 48 h post irradiation and subject to A) Gross images and histological evaluation exhibits that VitD protects from UV induced necrosis, dyskeratosis and vacuolation of the basal layer. B) treatment with VitD suppresses relative TNF-α, NOS2 and MMP9 48 h following UV exposure (n=6 for all groups, p<0.005 using a paired t-test.

FIGS. 16A-C provide graphs and images showing 25(OH)D enhances macrophage-specific autophagy. Ex Vivo staining of skin cell digest for detection of LC3 and F4/80 expression. Skin digested cells were plated on cover slips allowed to rest for 2 hours then fixed and stained for the specific targets. A) LC3+ punctae MFI in 25(OH)D treated mice compared to untreated mice exposed to UV only B) Immunofluorescent detection of F4/80 (green), LC3 (red) and DAPI (blue). C) Blockade of autophagy by inhibitor 3-MA deteriorated inflammation by increasing TNF-α and MMP9 significantly even after treatment with VitD.

FIGS. 17A-17E provide graphs and images showing VitD expands CD206+M2 macrophages in an autophagy dependent manner. Cells were harvested from skin and stained for detection of CD45 (myeloid), F4/80 (macrophage), CD206 (M2) and LC3 (marker for autophagy). Panels A-D—representative FACS dot plots indicating distribution of F480+CD45+ cells into 2 separate populations—CD206-Ly6C+ (M1) and CD206+Ly6C− (M2). Histogram showing LC3 count and MFI within the M1 and M2 population. Panel A—Control non irradiated skin cells show M2 to be 3-fold more than M1, LC3 MFI elevated 2-fold in M2 relative to M1, Panel B—UV expands M1 while depleting M2, LC3 is 2.7 fold higher in M2s but more M1 cells exhibit UV-induced autophagy, Panel C—VitD expands the M2 compared to M1 and panel D—Blockade of autophagy by inhibitor 3-MA recapitulates cellular profile observed with UV while quenching LC3 expression. E) Quantitative evaluation of flowcytometric analysis to show ratio of M2 to M1 indicating that VitD enhanced autophagy may be responsible for expansion of M2 macrophages to favor skin recovery from UV induced inflammation.

FIGS. 18A-18E provide graphs and images showing CD206-expressing macrophages in mouse skin tissue selectively enhance LC3 expression after treatment with 25(OH)D. A-D) Representative immunofluorescent images of mouse skin tissue co-localizing CD206 and LC3 predominantly after 25(OH)D treatment. E) Quantitation representation of CD206+LC3+ cells.

FIGS. 19A-19D provide images showing Vitamin D augments Arginase1 expression in M2 macrophages. A-B) UV irradiated skin sections show increased CD206+Arg1+ expression after treatment with VitD. C-D) inhibition of autophagy abrogates VitD induced Arg1 expression in M2 macrophages. Scale bar 20 μm.

FIG. 20 provides graphs and images showing VitD protects mice from UV induced apoptosis in an autophagy dependent manner. Irradiated skin tissue sections were subject to TUNEL staining to assess cellular apoptosis. A) Upper panel—Representative images show extensive Tunel positive cells in UV exposed skin that is significantly diminished by treatment with vitD. Lower Panel—Inhibition of autophagy worsens apoptosis in UV mice that cannot be reversed even after treatment with VitD. B) Quantitation of apoptosis in the skin. Bar scale: 100 μm.

FIG. 21 provides images showing Human irradiated skin exhibits macrophage specific autophagy that is enhanced by oral intervention with vitamin D. Images present immunofluorescently stained sections from a representative subject exposed to experimentally induced sunburn before after treatment with a single high dose of oral 200,000 IU D3. Images indicate enhanced LC3 (red) co-localized with CD163+(green) macrophages in treatment group compared to UV alone. Scale Bar 100 μm for 20× magnification and 20 μm for 63×.

FIGS. 22A-D provide graphs and images showing depletion of autophagy specifically in myeloid cells depletes M2 macrophages, reduces M2:M1 ratio and impairs VitD ability to attenuate inflammation. Atg7 KO LysMCre autophagy deficient mice were subjected to UV exposure preceding treatment with VitD. Cells were harvested from skin for flow cytometric analysis to show in Panel A) Littermates and atg7 KO controls exhibit a robust M2 population. Panels B,C) compared to littermates, autophagy deficient mice in presence or absence of VitD exhibit depleted CD206+M2 macrophages, thus D) M2:M1 ratio is comparable in presence or absence of VD in atg7 KO but significantly altered by VD among littermates.

### DETAILED DESCRIPTION OF THE INVENTION

The present invention provides a method of treating or preventing skin damage in a subject in need thereof that includes administering to the subject a therapeutically effective amount of an autophagy activator, such as vitamin D. The method can include administering doses of the autophagy activator that are substantially higher than those typically used.

### Definitions

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

The terms “comprising” and variations thereof do not have a limiting meaning where these terms appear in the description and claims.

The recitations of numerical ranges by endpoints include all numbers subsumed within that range (e.g., 1 to 5 includes 1, 1.5, 2, 2.75, 3, 3.80, 4, 5, etc.).

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

The term “therapeutically effective amount” can refer to the amount of a composition of the present application determined to produce any therapeutic response in a subject. For example, effective therapy decreases the level of skin damage and/or inhibits overt clinical symptoms resulting from the skin damage. Treatments that are therapeutically effective within the meaning of the term as used herein include treatments that minimize injury at a site of skin damage and/or promote healing at a site of skin damage and/or reduce or prevent swelling at a site of skin damage and/or improve a subject's quality of life. Such therapeutically effective amounts are readily ascertained by one of ordinary skill in the art. Thus, to “treat” means to deliver such an amount.

As used herein a “protective amount” refers to a nontoxic but sufficient amount of the composition used in the practice of the invention that is effective to decrease or prevent skin injury from exposure to a source of injury such as vesicants or radiation. That result can be reduction and/or alleviation of the signs, symptoms, or other results from exposure to sunlight and/or ultraviolet radiation. An appropriate protective amount in any individual case may be determined by one of ordinary skill in the art using routine experimentation.

A “subject,” as used herein, can be any animal, and may also be referred to as the patient. Preferably the subject is a vertebrate animal, and more preferably the subject is a mammal, such as a domesticated farm animal (e.g., cow, horse, pig) or pet (e.g., dog, cat). In some embodiments, the subject is a human. A subject in need of protection is a subject who is likely to be exposed to source of skin injury such as a vesicant or radiation in the near future.

Methods of Treating or Preventing Skin Damage

The present invention is directed to a method of treating or preventing skin damage in a subject in need thereof, comprising administering to the subject a therapeutically effective amount of an autophagy activator.

The term “skin damage” can refer to any type of skin damage is a skin burn injury that is caused by one or more of chemical burns caused, for example, by vesicants (e.g., weaponized vesicants, such as mustard gas, nitrogen mustard and sulfur mustard, and chemotherapeutic vesicants, such as mechlorethamine and doxorubicin), a radiation burn (e.g., radiotherapy/radiation therapy as part of a cancer treatment, or UV- or X-ray induced skin damage, including sunburn caused by sunlight), a heat burn (e.g., caused by fire, steam, hot objects or hot liquids), a cold burn, an electrical burn, and friction burn. UV-induced skin damage, for example, can refer to skin damage resulting from exposure to ultraviolet light in the A (320-400 nm), B (290-320 nm), or C ranges (200-290 nm). However, in other embodiments, the skin damage is caused by trauma, surgical and post-surgical wounds and wound healing, and other types of breakdown of the stratum corneum, epidermis, and underlying tissues. Skin damage can include, by way of example, erythema, edema, hyperpigmentation, dry desquamation, moist desquamation, epilation and ulceration.

The autophagy activator can be administered to the subject by the subject himself or herself, or by another person, e.g., a healthcare provider. The composition can be administered according to a prescribed treatment protocol (e.g., as determined by a healthcare professional) or as needed by a subject. In one example, skin damage can be reduced in the subject by at least 5% or more, at least 10% or more, at least 20% or more, at least 25% or more, at least 30% or more, at least 35% or more, at least 40% or more, at least 45% or more, at least 50% or more, at least 55% or more, at least 60% or more, at least 65% or more, at least 70% or more, at least 75% or more, at least 80% or more, at least 85% or more, at least 90% or more, at least 95% or more, or entirely (100%). In some instances, a reduction in skin damage can be assessed based on the measured induction of repithelialization (i.e., the generation of new cells in the epithelium) or the measured decrease in wound area.

In some embodiments, the skin damage is the result of radiation exposure, such as exposure to harsh sunlight. Tissue injury resulting from exposure to sunlight is also known as “sunburn.” Typically, symptoms of sunburn include initial redness (erythema), followed by varying degrees of pain, proportional in severity to both the duration and intensity of exposure. Other symptoms can include edema, itching, peeling skin, rash, nausea, fever, chills, and syncope. The symptoms of sunburn represent a reaction of the body to DNA damage. Accordingly, protecting a subject from skin damage from chemical or radiation burns can also decrease the likelihood that the subject will develop cancer, and in particular skin cancer (e.g., melanoma).

In other embodiments, the skin damage is caused by a burn-causing chemical such as a vesicant. A vesicant is a chemical compound that causes severe skin, eye and mucosal pain and irritation, resulting in painful water blisters on affected subjects. Vesicants include naturally toxic agents, harmful industrial chemicals, and chemical warfare agents. Examples of natural vesicants include cantharidin and furanocoumarin. Examples of chemical warfare vesicants include mustards (sulfur mustards and nitrogen mustards), Lewisite (2-chloroethenylarsonous dichloride), and phosgene oxime.

As used herein, the term “protection” refers to a decrease in skin damage, tissue injury and/or symptoms of tissue injury resulting from exposure of the skin to sources of injury, such as radiation and vesicants. The decrease in damage or tissue injury and/or symptoms can vary in degree. For example, in some embodiments, the decrease is at least a 50% decrease, at least a 60% decrease, at least a 70% decrease, at least an 80% decrease, at least a 90% decrease, or a 100% decrease, which can also be referred to as prevention of injury.

The method includes administering a therapeutically effective amount of an autophagy activator to a subject. “Autophagy,” as used herein, refers to a variety of tightly-regulated catabolic processes that involve the degradation of a cell's own components through the lysosomal machinery and play a normal part in cell growth, development, and homeostasis, helping to maintain a balance between the synthesis, degradation, and subsequent recycling of cellular products. The most well-known catabolic process of autophagy involves the formation of a membrane around a targeted region of the cell, separating the contents from the rest of the cytoplasm. The resultant vesicle then fuses with a lysosome and subsequently degrades the contents.

Autophagy can be induced by many factors both from within and outside the cell, including starvation, nutrient deprivation, bacterial infection, damage to cellular organelles, and protein mismatching. Starvation-induced autophagy is the best understood mechanism for autophagy activation. However, it has been demonstrated that a number of intracellular signaling molecules, such as AMPK, mTOR, C3PI3K, and MAPK, are also involved in autophagy regulation. A basic biochemical reaction that mediates the formation of the autophagic (isolation) membrane is catalyzed by a conserved kinase, PI3K-III (type III phosphatidylinositol 3-kinase). This enzyme converts phosphatidyl-inositol-3 phosphate into phosphatidyl-inositol-3,5 bisphosphate. Thus, PI3K-III is a critical component of the autophagic process. The molecular antagonists of PI3K-III involve certain myotubularin-related (MTMT) phosphatases. These MTMT enzymes can inhibit autophagic degradation. In genetic model systems and cell cultures, inhibition of mtm genes leads to a potent autophagy activation. Loss-of-function mutations in mtm genes can significantly extend lifespan, suppress neuronal cell death, and prevent muscle and other tissues from undergoing atrophy. A myotubularin protein (MTMT14) is implicated in fine tuning of autophagy.

The term “autophagy activator,” as used herein, refers to any agent, compound, or moiety capable of promoting and/or inducing autophagy in a cell. Whether or not an agent, compound, or moiety is an autophagy activator, or has autophagy-activating effects (e.g., in vitro or in vivo), can be assessed, for example, by evaluating the capacity or efficacy of the agent, compound, or moiety for clearance in cells; in other words, the autophagy activity of the agent, compound, or moiety. Accordingly, autophagy activators also include compounds identified by the screening methods in which compounds are assessed for their activity on enzymes important for autophagy, or on autophagy itself. See U.S. Patent Publication No. 2012/01788119 for examples of methods to assess autophagy activity. When autophagy activity is higher, the clearance is regarded as functioning in living cells.

A variety of autophagy activators are known to those skilled in the art. For example, the autophagy activator can be an mTOR pathway inhibitor (e.g., rapamycin,), Vitamin D, a Vitamin D analogue, a pharmaceutically active source of Vitamin D, or an mTOR pathway-independent autophagy activator (e.g., trehalose). Autophagy activators also include seven compounds that have already been approved by the FDA and one compound with known Ca²⁺ channel activity. These compounds can promote the degradation of long-lived proteins within the cell and reduce over-expression levels of polyQ in transfected cells. These seven additional compounds include Loperamide, Amiodarone, Niguldipine, Pimozide, Nicardipine, Penitrem A, Fluspirilene, and Trifluoperazine. See U.S. Patent Publication No. 2017/0050929. Others have shown that myotubularin protein (MTMT14) inhibitors are autophagy activators (See U.S. Patent Publication No. 2016/0346283) and Beclin 1 peptide analogs can be used as autophagy activators (See U.S. Patent Publication No. 2015/0359840).

In some embodiments, the autophagy activator is vitamin D, a vitamin D analog, or a vitamin D metabolite. Vitamin D compounds are fat-soluble seco-steroid precursors to vitamin D prohormones that contribute to the maintenance of normal calcium and phosphorus levels in the bloodstream. The term “vitamin D” includes ergocalciferol (vitamin D2) and cholecalciferol (vitamin D3) as well as to their metabolites and analogs. Vitamin D3 metabolites include alfacalcidol (1hydroxycholecalciferol), calcitriol (1α,25-dihydroxycholecalciferol), dihydrotachysterol, while examples of Vitamin D2 metabolites include 1α,25-dihydroxyvitamin D2 and 1α,24(S)-dihydroxyvitamin D2. Vitamin D analogs are described in U.S. Pat. No. 4,851,401 (cyclopentano-vitamin D analogs), U.S. Pat. No. 5,120,722 (trihydroxycalciferol derivatives), U.S. Pat. No. 5,446,035 (20-methyl substituted vitamin D), U.S. Pat. No. 5,411,949 (23-oxa-derivatives), U.S. Pat. No. 5,237,110 (19-nor-vitamin D compounds), U.S. Pat. No. 4,857,518 (hydroxylated 24-homo-vitamin D derivatives). Additional Vitamin D analogs are taught in U.S. Pat. Nos. 4,804,502; 4,866,048; 5,145,846 5,374,629; 5,403,940; 5,446,034; and 5,447,924, among others.

In some embodiments, a high dose of the autophagy activating agent is administered to the subject. A high dose is one that is substantially larger than the dose of the compound that is typically administered to the subject. A higher dose can be one that is greater than the average typical dose, 2× greater than the average typical dose, 5× greater than the average typical dose, or 10× or more greater than the average typical dose. Typical average doses of autophagy activators are known to those skilled in the art. For example, a high dose of vitamin D includes administering at least 50,000 IUs of the vitamin D, vitamin D analog, or vitamin D metabolite are administered to the subject. In other embodiments, a high dose of vitamin D includes administering at least 100,000 IUs of the vitamin D, vitamin D analog, or vitamin D metabolite are administered to the subject, while in a further embodiments, at least 200,000 IUs of the vitamin D, vitamin D analog, or vitamin D metabolite are administered to the subject. Dosages of the substance of the present invention can vary between wide limits, depending upon a variety of factors including the disease or disorder to be treated, the age, weight and condition of the individual to be treated, the route of administration etc.

In some embodiments, animal models can be used to determine the appropriate dosage for an autophagy activating agent in human subjects. For example, the dosage of an oral administration of cholecalciferol in humans can be correlated with the dosage of an intraperitoneal injection of 25(OH)D in mice to obtain useful dosages.

### Pharmaceutical Compositions

The autophagy activator can be administered as part of a pharmaceutical composition. The pharmaceutical composition may additionally comprise a pharmaceutically acceptable excipient for example a pharmaceutically acceptable carrier and/or a pharmaceutically acceptable diluent. Suitable carriers and/or diluents are well known in the art and include pharmaceutical grade starch, mannitol, lactose, magnesium stearate, sodium saccharin, talcum, cellulose, glucose, sucrose (or other sugar), magnesium carbonate, gelatin oil, alcohol, detergents, emulsifiers or water (preferably sterile).

A pharmaceutical composition may be provided in unit dosage form, will generally be provided in a sealed container and may be provided as part of a kit. Such a kit would normally (although not necessarily) include instructions for use. It may include a plurality of said unit dosage forms.

A pharmaceutical composition may be adapted for administration by any appropriate route, for example by the oral (including buccal or sublingual), rectal or topical (including buccal, sublingual or transdermal) route. Such compositions may be prepared by any method known in the art of pharmacy, for example by admixing the active ingredient with a carrier(s) or excipient(s) under sterile conditions.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc. For the preparation of solutions and syrups, excipients which may be used include for example water, polyols and sugars. For the preparation of suspensions oils (e.g. vegetable oils) may be used to provide oil-in-water or water in oil suspensions.

Pharmaceutical compositions adapted for topical administration may be formulated as ointments, creams, suspensions, lotions, powders, solutions, pastes, gels, sprays, aerosols or oils. For infections of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes. Pharmaceutical compositions adapted for rectal administration may be presented as suppositories or enemas.

Pharmaceutical compositions adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators. Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water, alcohols, polyols, glycerine and vegetable oils, for example. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

The pharmaceutical compositions may contain preserving agents, solubilising agents, stabilising agents, wetting agents, emulsifiers, sweeteners, colourants, odourants, salts, buffers, coating agents or antioxidants. They may also contain an adjuvant and/or therapeutically active agents in addition to the autophagy activator.

Monitoring the Effects of an Autophagy Activator on Skin Damage Using Arginase-1

Another aspect of the invention provides a method of monitoring the immunomodulatory effects of an autophagy activator on skin damage in a subject. The method includes administering the authophagy activator to the subject and determining the levels of arginase-1 in a biological sample obtained from the subject, wherein an increased level of arginine-1 indicates the autophagy activator is having an effective immunodulatory effect. For example, increased skin expression of the anti-inflammatory mediator arginase-1 is associated with recovery from skin damage. Arginase-1 is a known urohydrolase enzyme that converts L-arginine into L-ornithine and urea. The autophagy activator being administered can be any autophagy activator as described herein. For example, in some embodiments, the autophagy activator is vitamin D. The skin damage can be the result of any of the causes of skin damage described herein, such as exposure to a vesicant or damaging radiation.

Immunomodulation refers to an effect on the immune system of the subject. Examples of immunomodulation include immunostimulation and immunosupresssion. Immunomodulation can occur as a result of an effect on cells of the immune system (e.g., macrophages), and/or as a result of an effect on products of the immune system, such as antibodies or cytokines.

The method of monitoring the immunomodulatory effect of autophagy activators involves determining the level of arginase-1 in a biological sample. Biological samples include, but are not necessarily limited to bodily fluids such as saliva, urine and blood-related samples (e.g., whole blood, serum, plasma, and other blood-derived samples), cerebral spinal fluid, bronchoalveolar lavage, and the like. In some embodiments, the biological sample is a skin sample. Biological samples can be obtained by any known means including needle stick, needle biopsy, swab, and the like.

A biological sample may be fresh or stored (e.g. blood or blood fraction stored in a blood bank). Samples can be stored for varying amounts of time, such as being stored for an hour, a day, a week, a month, or more than a month. The biological sample may be a bodily fluid expressly obtained for the assays of this invention or a bodily fluid obtained for another purpose which can be sub-sampled in order to carry out the method.

The present invention is illustrated by the following examples. It is to be understood that the particular examples, materials, amounts, and procedures are to be interpreted broadly in accordance with the scope and spirit of the invention as set forth herein.

### EXAMPLES

### Example 1: Oral Vitamin D Rapidly Attenuates Inflammation from Sunburn

The inventors designed a pilot, proof-of-principle interventional study in humans, modeled after a randomized, double-blinded, placebo-controlled clinical trial, to test the hypothesis that a single high dose of oral vitamin D3 (cholecalciferol) would be capable of rapidly attenuating experimental sunburn induced by simulated solar radiation (SSR).

### Results

Dose-Dependent Response of High Dose Oral Vitamin D3 and UV Irradiation

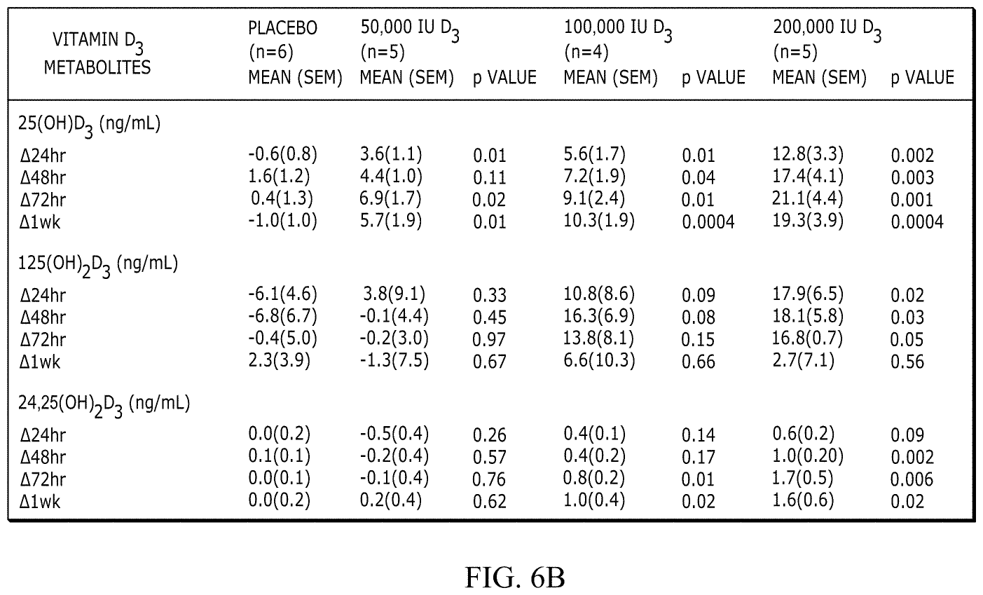

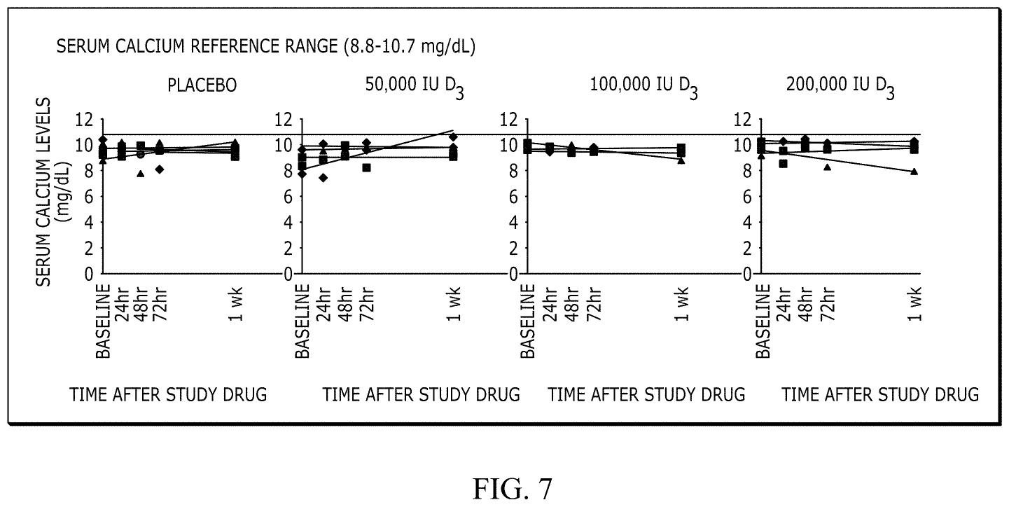

The randomized treatment groups did not differ in their baseline characteristics (Table 1). No participant was taking supplemental vitamin D3 before study initiation. Serum 25-hydroxyvitamin D3 (25(OH)D₃), a marker of vitamin D3 stores, increased after treatment in a vitamin D3 dose-dependent fashion (FIG. 6A). Similar trends were observed for the active form of vitamin D3, 1,25(OH)₂D₃, as well as an inactive breakdown product, 24,25(OH)₂D₃ (FIG. 6B). No measured vitamin D3 metabolite increased into a toxic range in any of the treatment groups throughout the study period. Furthermore, there were no instances of clinically significant hypercalcemia occurring in any of the treatment groups throughout the study period (FIG. 7).

TABLE 1

| | Placebo | | 50,000 IU D₃ | | 100,000 IU D₃ | | 200,000 IU D₃ | |
|---|---|---|---|---|---|---|---|---|
| | (n = 6) | | (n = 5) | | (n = 4) | | (n = 5) | |
| Age, median (range) | 24.0 | (21-46) | 35.0 | (21-58) | 36.5 | (22-50) | 27.0 | (21-53) |
| Sex, N (%) | | | | | | | | |
| Male | 3 | (50.0) | 3 | (60.0) | 3 | (75.0) | 4 | (80.0) |
| Female | 3 | (50.0) | 2 | (40.0) | 1 | (25.0) | 1 | (20.0) |
| BMI, median (range) | 22.8 | (16.1-26.8) | 23.1 | (21.3-31.9) | 28.6 | (22.6-33.1) | 23.0 | (21.7-41.6) |
| FST, N (%) | | | | | | | | |
| I | 0 | (0.0) | 0 | (0.0) | 0 | (0.0) | 1 | (20.0) |
| II | 2 | (33.3) | 3 | (60.0) | 2 | (50.0) | 3 | (60.0) |
| III | 4 | (66.6) | 2 | (40.0) | 2 | (50.0) | 1 | (20.0) |
| Baseline Vitamin D₃ | | | | | | | | |
| Metabolites, | | | | | | | | |
| mean (95% CI) | | | | | | | | |
| 25(OH)D₃ (ng/mL) | 26.8 | (23.3, 30.4) | 30.9 | (21.1, 40.6) | 18.6 | (10.8, 26.4) | 22.4 | (15.4, 29.4) |
| 1,25(OH)₂D₃ (pg/mL) | 61.8 | (47.1, 76.6) | 54.5 | (50.3, 58.8) | 57.7 | (46.1, 69.4) | 54.8 | (42.4, 67.2) |
| 24,25(OH)₂D₃ (ng/mL) | 2.4 | (1.8, 3.0) | 3.0 | (1.5, 4.5) | 1.4 | (0.9, 1.8) | 1.8 | (0.9, 2.6) |

Abbreviations:
D, vitamin D₃ (cholecalciferol);
BMI, body mass index (the weight in kilograms divided by the square of the height in meters);
FST, Fitzpatrick skin type;
CI, confidence interval
*There were no statistically significant differences in the treatment groups at baseline.

Additional experiments were carried out demonstrating the effect of high doses of vitamin D on erythema and edema. The results are shown in Table 2.

TABLE 2
Vitamin D effects on erythema and edema
Erythema and edema

| Placebo | 50,000 IU D₃ | | 100,000 IU D₃ | | 200,000 IU D₃ | |
|---|---|---|---|---|---|---|
| (N = 6) | (N = 5) | | (N = 4) | | (N = 5) | |
| Mean | Mean | p | Mean | p | Mean | p |
| (95% CI) | (95% CI) | value | (95% CI) | value | (95% CI) | value |

Erythema

| | | | | | | | |
|---|---|---|---|---|---|---|---|
| Δa*_{24 hr} | 3.6 (−1.0.8.3) | 1.4 (−4.4.7.3) | 0.57 | 2.0 (−2.1.6.1) | 0.63 | 0.1 (−2.5.2.6) | 0.24 |
| Δa*_{48 hr} | 4.1 (−0.8.9.0) | 2.3 (−3.6.8.2) | 0.65 | 2.4 (−3.2.8.0) | 0.67 | −0.5 (−3.3.2.3) | 0.17 |

Thickness

| | | | | | | | |
|---|---|---|---|---|---|---|---|
| Δth_{72 hr} | 0.3 (−0.1.0.8) | 0.1 (−0.5.0.7) | 0.65 | −0.7 (−0.9.−0.5) | 0.03 | −0.5 (−0.9.0.0) | 0.09 |
| Δth_{1 wk} | 0.2 (−0.3.0.7) | 0.3 (−0.2.0.8) | 0.81 | −1.0 (−1.7.−0.3) | 0.02 | −0.4 (−1.9.1.0) | 0.37 |

Abbreviations:
IU—international units;
D₃—vitamin D₃ (cholecalcifierol);
CI—confidence interval;
MED—minimal erythema dose;
a*—skin erythema as measured by chromameter;
th—skin thickness as measured by skin caliber
Note:
Changes in erythema and thickness refer to irradiation a 2MED and 3MED, respectively. Statistical comparisons were made between the vitamin D treatment groups and the placebo group.

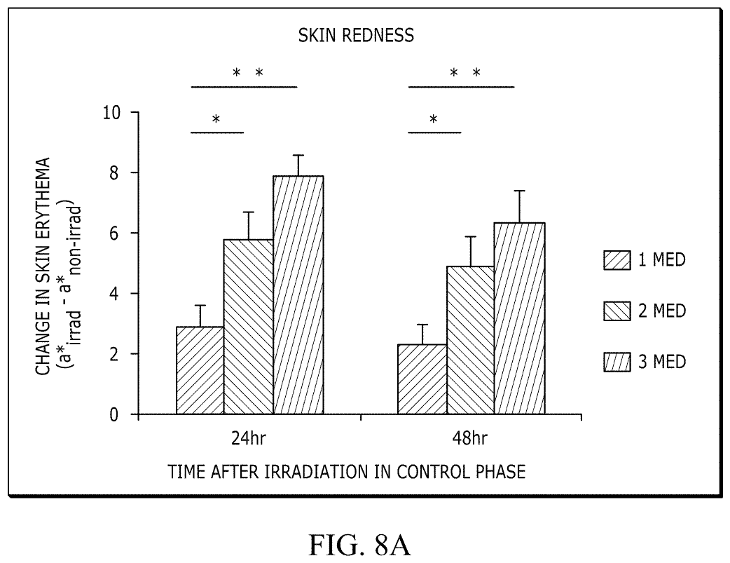

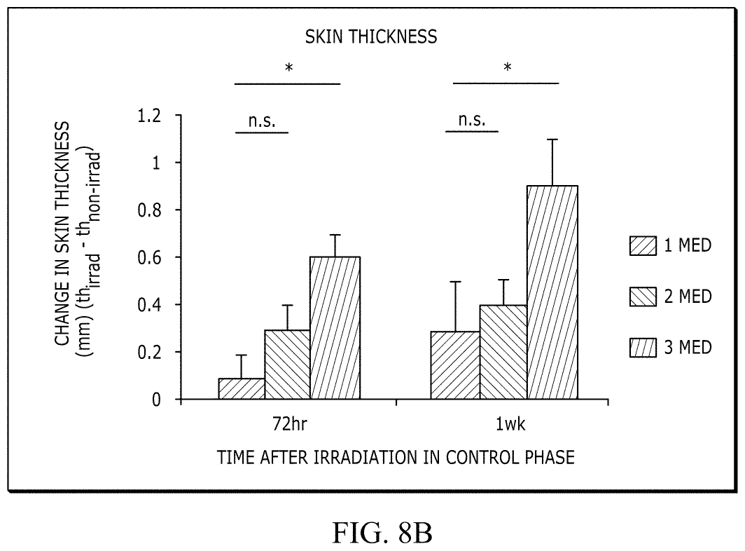

Sunburn is a stereotypical inflammatory response induced by exposure to an erythemogenic dose of UVR. Sunburn is characterized clinically by redness, mediated by dermal vasodilatation, and edema, mediated by increased vascular permeability and inflammatory cell infiltration. Clydesdale et al., Immunology and cell biology, 79(6):547-68 (2001). While skin redness peaks early after UVR exposure, skin thickness increases steadily for up to two weeks after irradiation. Ouhtit et al., Am J Pathol., 156(1):201-7 (2000). Compared to one minimal erythema dose (MED), exposure to 2MED increased skin erythema 24 hr and 48 hr after irradiation, and exposure to 3MED increased skin thickness 72 hr and 1 week after irradiation (p<0.05 for all) (FIGS. 8A & 8B). We observed saturation of skin redness after exposure to 3MED, limiting the ability to discern subtle differences among treatment groups with this high dose of UVR.

High Dose Oral Vitamin D3 Attenuates Skin Inflammation

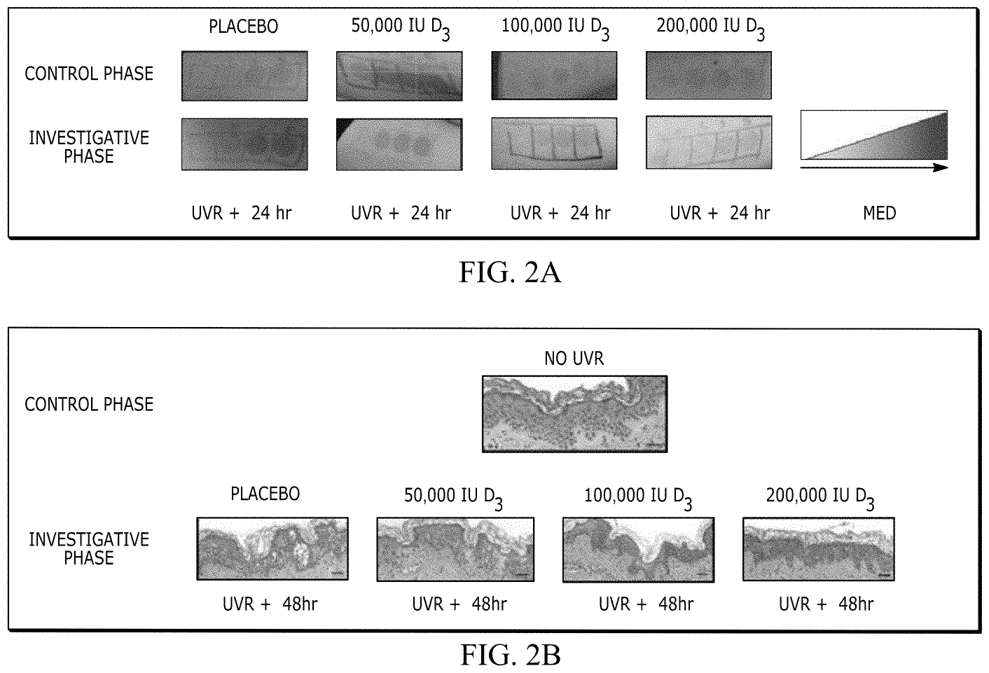

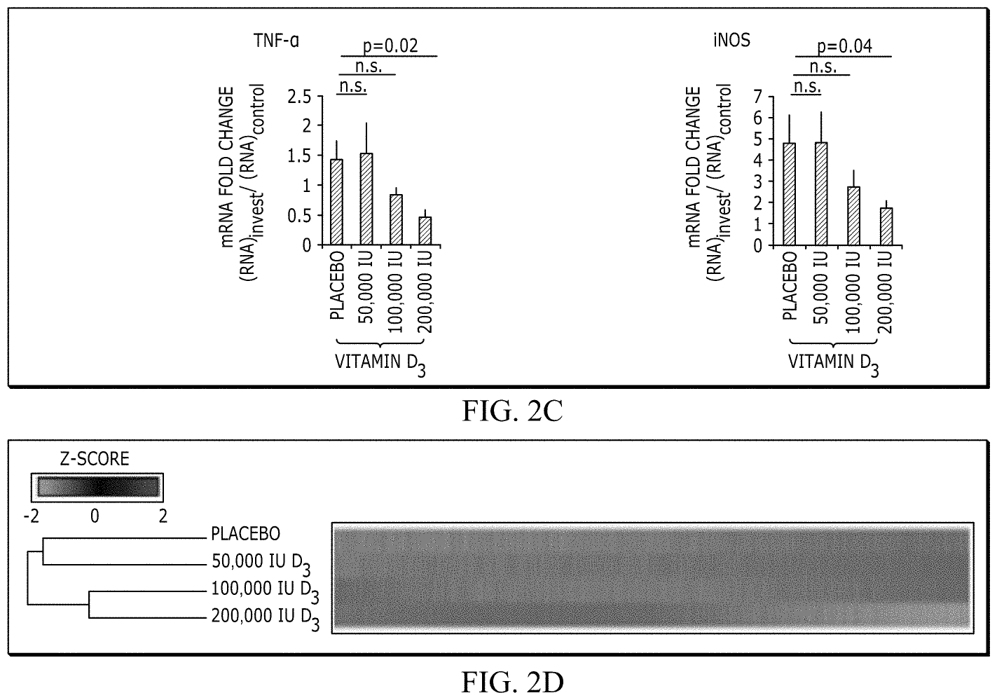

Clinically, irradiated skin appeared red and swollen 48 hr after UVR exposure (FIG. 2A). Irradiated skin also displayed histologic evidence of structural damage as compared to non-irradiated skin, including epidermal vesiculation and edema formation, which improved in a vitamin D3 dose-dependent fashion (FIG. 2B). Skin expression of TNF-α and iNOS was lower in participants receiving 200,000 international units (IU) D3 as compared to placebo 48 hr after irradiation (p=0.04 for TNF-α; p=0.02 for iNOS) (FIG. 2C). With higher doses of vitamin D3, there was a trend for decreased skin thickness after irradiation, which reached significance in the 100,000 IU D3 group at both 72 hr (p=0.03) and 1 week (p=0.02) after irradiation. Comparison of global gene expression profiles among the treatment groups revealed that the 200,000 IU D3 group had a distinct gene expression profile that was very different from the placebo group, and most closely related to the 100,000 IU D3 group (FIG. 2D).

Elevated Serum Levels of 25(OH)D₃ Correlate with Decreased Skin Redness

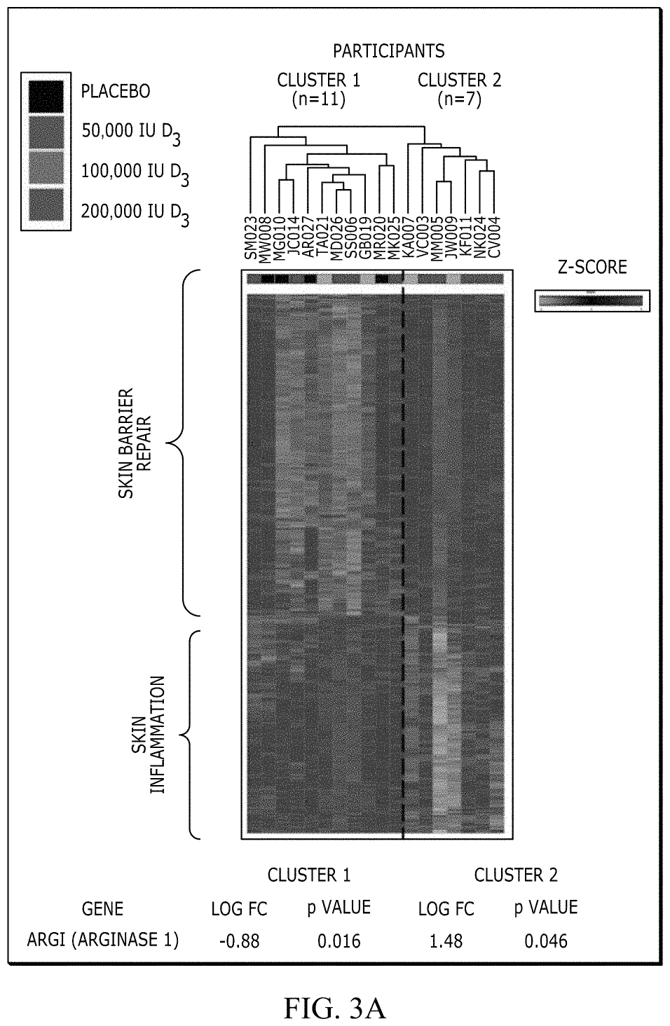

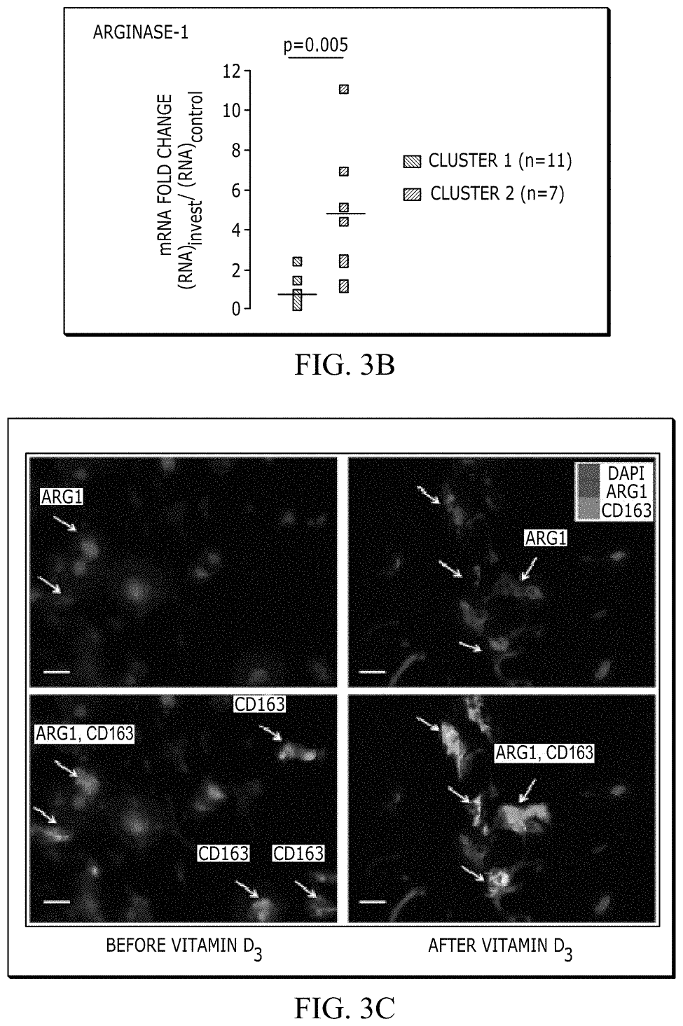

To further investigate a potential link between vitamin D3 and gene expression, we analyzed the global gene expression profiles of all participants, blinded to their allocated treatment groups. The dendrogram resulting from this analysis produced two clusters of participants (FIG. 3A), representing an unbiased, unsupervised hierarchical clustering of all individuals based on similarities in gene expression profiles.

Of note, arginase-1, known to enhance tissue repair and inhibit inflammation through the utilization of iNOS precursors, was significantly down regulated in cluster 1 (p=0.016) and up regulated in cluster 2 (p=0.046) (FIG. 3A). Bronte V, Zanovello P., Nat Rev Immunol., 5(8):641-54 (2005). Increased arginase-1 expression observed in cluster 2 compared to cluster 1 was subsequently validated with qRT-PCR (p=0.005) (FIG. 3B). Additionally, confocal microscopy analysis of a representative participant from cluster 2 revealed increased expression of the arginase-1 protein localized to CD163+ macrophages after vitamin D3 treatment (FIG. 3C).

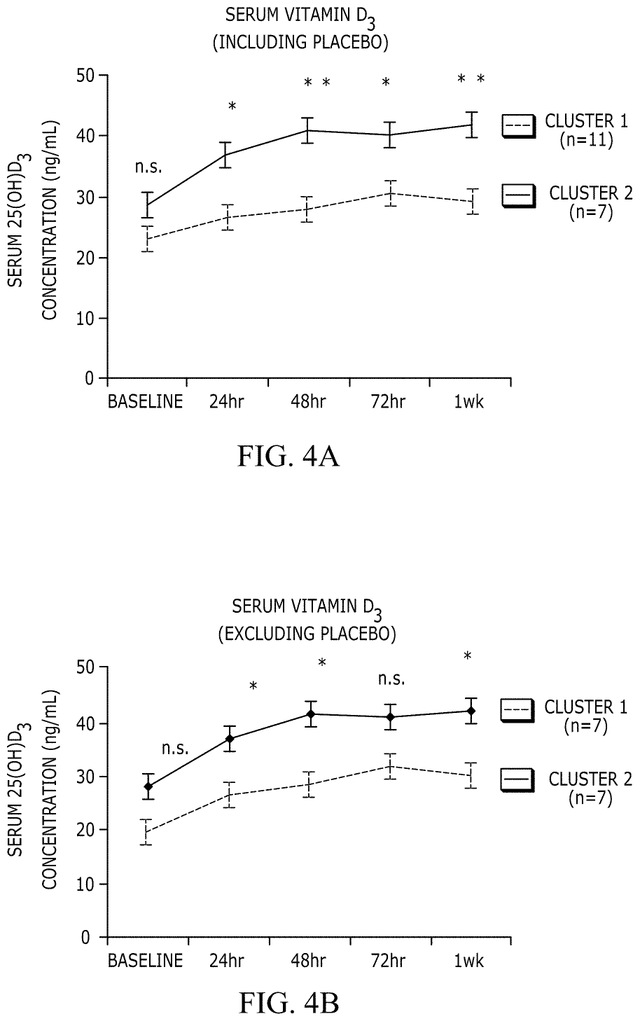

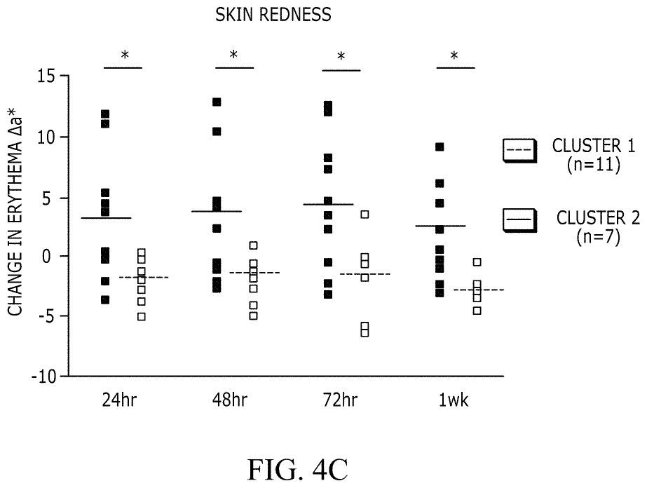

Unblinding of the participants' demographics and treatment group allocation revealed that the two clusters of participants did not differ in their baseline characteristics. However, cluster 1 contained all participants randomized to receive placebo, as well as a mixture of participants from the various vitamin D3 treatment groups (FIG. 3A). Cluster 2 predominately contained participants who had received higher doses of vitamin D3, and notably none of the participants who had received placebo. While the two clusters had similar baseline serum 25(OH)D₃ levels, participants in cluster 2 had significantly higher 25(OH)D₃ levels after treatment as compared to participants in clusters 1 (p<0.05 for all time points) (FIG. 4A). When subjects receiving placebo were excluded from this analysis, serum 25(OH)D₃ levels after treatment remained lower for participants in cluster 1 as compared to participants in cluster 2 (p<0.05 for 24 hr, 48 hr and 1 week) (FIG. 4B). We will now refer to participants from cluster 2 as vitamin D3 responders and participants from cluster 1 as vitamin D3 non-responders.

As indicated above, body-mass index (BMI), age, gender, and baseline 25(OH)D₃ stores had no effect on the serum response to oral vitamin D3. Furthermore, along with higher serum 25(OH)D₃ levels, vitamin D3 responders demonstrated a statistically significant sustained reduction in skin redness at all time points after irradiation as compared to vitamin D3 non-responders (p<0.05 for all) (FIG. 4C), and a trend for reduced skin thickness 1 week after irradiation.

Differential Gene Expression Profiles Characterize Vitamin D3 Responders and Vitamin D3 Non-Responders

Vitamin D3 non-responders displayed up regulation of various pro-inflammatory genes not observed in the gene expression profiles of vitamin D3 responders, including matrix metalloproteinases (MMP1, MMP3), interleukin-1 alpha (IL-1A), and monocyte chemokines (CCL2). Likewise, canonical pathways related to leukocyte migration and IL-6 signaling were significantly activated in vitamin D3 non-responders, including TNF-α as a predicted up-stream regulator. Conversely, vitamin D3 non-responders displayed a strikingly different gene expression profile, characterized by up regulation of genes implicated in skin barrier repair, including tissue transglutaminases (TGM3, TGM5), keratins (KRT78, KRT80), corneodesmosin (CDSN), and calmodulin-like 5 (CALML5).

### Discussion

In this pilot, proof-of-principle human interventional study modeled after a randomized, double-blinded, placebo-controlled trial, we provide in vivo evidence that a single high dose of oral vitamin D3 is capable of rapidly attenuating a local inflammatory response to UVR. Participants responding to high doses of vitamin D3 demonstrated a sustained reduction in skin redness following experimental sunburn, as well as less epidermal structural damage, reduced expression of pro-inflammatory markers in the skin, and a gene expression profile characterized by up regulation of skin barrier repair genes. This study also demonstrates that regardless of baseline serum vitamin D3 levels, a single high dose of oral vitamin D3 is safe, with serum vitamin D3 and calcium concentrations remaining within a normal reference range. Rosen C J., N Engl J Med 364(3):248-54 (2011).

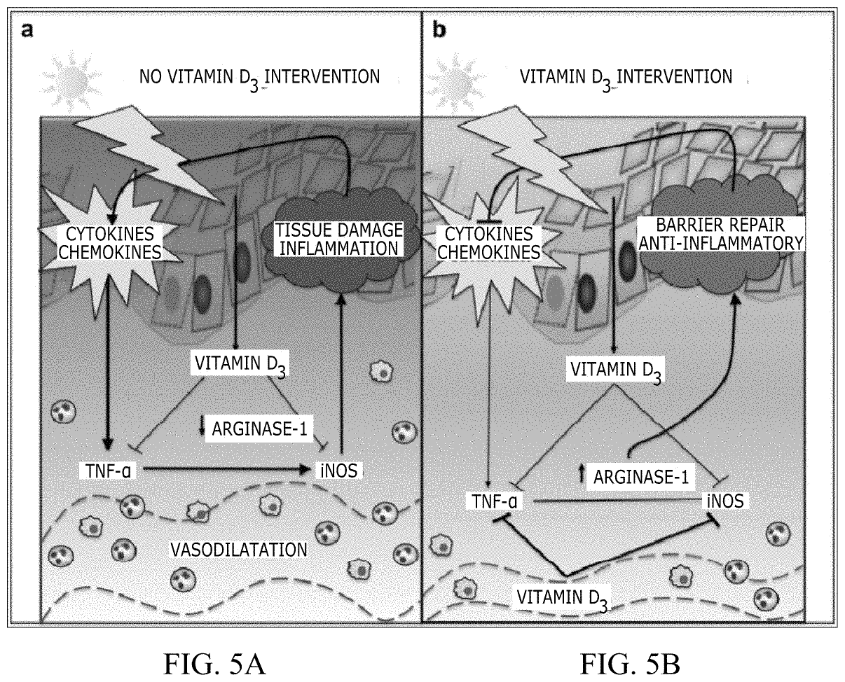

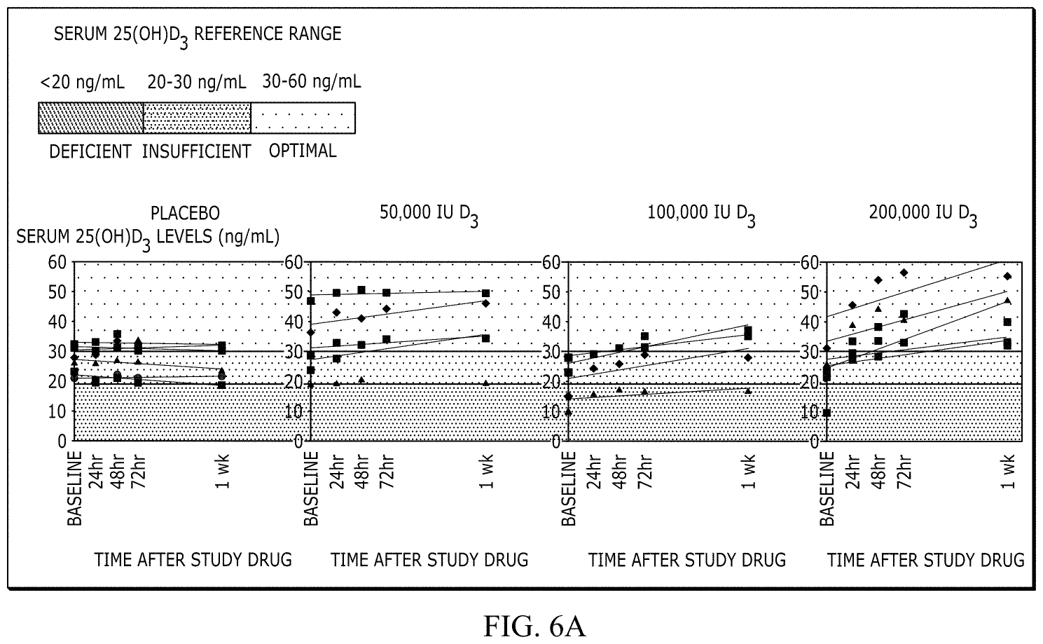

Moreover, up-regulation of arginase-1 is associated with the anti-inflammatory effects of vitamin D3 in humans (FIG. 5). While arginase has been identified to be present at physiological levels in inflammatory skin diseases, tumors, and chronic wounds, to our knowledge the induction of arginase-1 expression by vitamin D3 in human skin in vivo is previously unreported. Bruch-Gerharz et al., Am J Pathol., 162(1):203-11 (2003). These findings suggest that arginase-1 may also be a clinically useful tissue biomarker for monitoring the immunomodulatory effects of vitamin D3 in humans. Given the presence of a putative vitamin D3 response element upstream of the arginase-1 promoter, future studies should be aimed at defining the mechanism by which vitamin D3 treatment activates the arginase-1 pathway. Andrukhova et al., Mol Endocrinol., 28(1):53-64 (2014).

Exploratory analyses suggest that the host's response to vitamin D3 intervention plays a critical role in the modulation of inflammation. Participants segregated into two clusters based on similarities in global gene expression profiles, and these two clusters differed significantly in their serum vitamin D3 levels after treatment. The pharmacokinetic properties of oral vitamin D3 are complex, however, and an individual's serum response to oral vitamin D3 depends on the dose of vitamin D3, age, BMI, baseline vitamin D3 stores, and genetic polymorphisms. Didriksen et al., Eur J Endocrinol., 169(5):559-67 (2013); Ilahi et al., Am J Clin Nutr., 87(3):688-91 (2008). Large randomized, double-blinded, placebo-controlled trials will be required to elucidate factors responsible for the therapeutic variability of vitamin D3 treatment within populations and to determine whether individuals with lower baseline vitamin D3 levels require higher treatment doses to achieve immunomodulation.

Exposure to erythemogenic doses of UVR initiates an influx of inflammatory cells into the skin, generating a microenvironment rich in inflammatory mediators. Cooper et al., J Invest Dermatol., 101(2):155-63 (1993). Specifically, release of TNF-α by damaged keratinocytes and other inflammatory cells plays a crucial role in initiating and sustaining UVR-induced inflammation. Following an inflammatory insult, classically activated M1-polarized macrophages infiltrate the skin and produce iNOS as part of an oxidative burst in an evolutionarily conserved attempt to prevent infection. Bronte V, Zanovello P., Nat Rev Immunol., 5(8):641-54 (2005). However, excessive production of iNOS perpetuates tissue damage, retards the resolution of inflammation, and prevents tissue repair. Das et al., J Invest Dermatol., 135(2):389-99 (2015). We and others have shown using murine models that vitamin D3 inhibits the production of TNF-α, and is capable of attenuating skin inflammation by reducing macrophage-specific iNOS production. Au et al., J Invest Dermatol. 135(12):2971-81 (2015); Zhang et al., J Immunol., 188(5):2127-35 (2012).

In the presence of retinoic acid, vitamin D3 induces the in vitro differentiation of monocytes into alternatively activated, M2-polarized CD163+ macrophages expressing arginase-1. Di Rosa et al., Cellular immunology, 280(1):36-43 (2012). Furthermore, it has been shown that exposure to acute UVR increases endogenous retinoids in the skin of mice. Gressel et al., Photochem Photobiol., 91(4):901-8 (2015). Taken together, our results combined with these data suggest that a potential mechanism by which vitamin D3 mediates resolution of experimental sunburn is via the up regulation of arginase-1 by endogenous repair molecules, leading to the selective induction of anti-inflammatory, M2-polarized CD163+ macrophages. Additionally, vitamin D3 may have other protective mechanisms in skin, including reducing DNA damage and keratinocyte apoptosis following experimental sunburn, as was shown in mice treated topically with the active form of vitamin D3 immediately after exposure to UVR. Dixon et al., J Steroid Biochem Mol Biol., 103(3-5):451-6 (2007).

It is likely that the generation of vitamin D3 from cholesterol precursors in the skin after UVR evolved to perform vital homeostatic functions. Moreover, skin-resident cells are capable of locally converting vitamin D3 into its active form, which can then signal in an intracrine, autocrine, and paracrine fashion to exert diverse biological effects. It is worthwhile to conjecture that vitamin D3 may provide an “endocrine barrier” within the skin, utilizing energy derived from sunlight to reduce inflammation, and promote wound healing, tissue repair, and an enhanced epidermal barrier. This would provide the host with additional protection against environmental insults by complementing the classically described brick and mortar mechanical, melanin pigment, and Langerhans cell immunologic barriers.

### Materials and Methods

Screening, Randomization, and Study Design

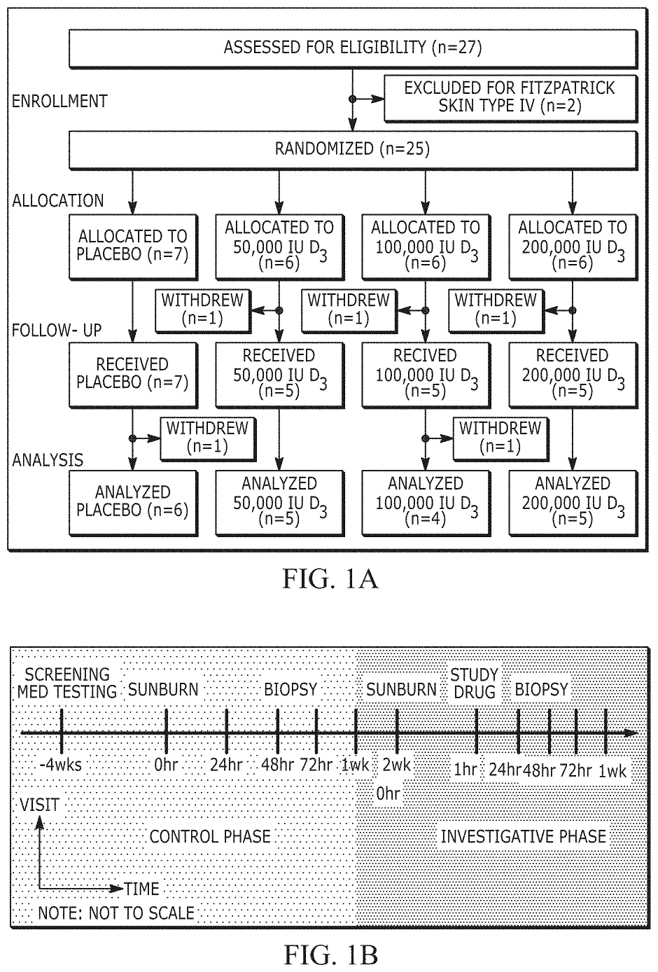

The Institutional Review Board at University Hospitals Cleveland Medical Center approved this pilot study, which was conducted between March 2013 and February 2015. The study was modeled after a randomized, double-blinded, placebo-controlled trial. Twenty-seven healthy adults 18 years and older were screened for eligibility and provided written informed consent (FIG. 1A). A total of twenty-five participants were randomized to receive, in a double-blinded fashion, either placebo or a single oral dose of vitamin D3 (cholecalciferol) at 50,000, 100,000, or 200,000 IU one hour after SSR exposure. UVR was administrated as SSR emitted from a 1000 watt Xenon arc lamp (Newport, Stratford, Conn.), a full spectrum light source that closely resembles natural sunlight.

This study was designed in a parallel fashion with exposure to SSR occurring on one arm without study drug administration (control phase), followed two weeks later by exposure to SSR on the contralateral arm with study drug administration (investigative phase) (FIG. 1B). The MED to experimentally induce sunburn was determined for each participant during the initial screening visit as previously described. Heckman et al. J Vis Exp., (75):e50175 (2013). Participants were irradiated with one, two, and three times the MED on the sun-shielded, upper arm using plastic holed templates to ensure that adjacent skin was not exposed. A total of twenty participants completed both phases of the study and were included in the per-protocol analysis.

Quantification of Vitamin D3 Metabolites and Calcium

The concentrations of the vitamin D3 metabolites 25(OH)D₃, 1,25(OH)₂D₃, and 24,25(OH)₂D₃, as well as total serum calcium, were measured from freshly frozen serum obtained during the screening visit, as well as 24 hr, 48 hr, 72 hr, and 1 week after receiving the study drug. Serum levels of 25(OH)D₃ (ng/mL) and 1,25(OH)₂D₃ (pg/mL) were measured by Liaison assay, and serum levels of 24,25(OH)₂D₃ (ng/mL) were measured by liquid chromatography-mass spectrometry (Heartland Assays, Ames, Iowa). Toxic serum 25(OH)D₃ levels were defined as those greater than 150 ng/mL. Total serum calcium was measured by the University Hospitals Cleveland Medical Center core laboratory (Cleveland, Ohio), and the normal reference range was considered 8.8 to 10.7 mg/dL.

Primary Outcomes of Randomized Participants

Primary outcomes included non-invasive measurements of skin erythema and thickness 24 hr, 48 hr, 72 hr, and 1 week after irradiation, as well as tissue expression of TNF-α and iNOS 48 hr after irradiation. Skin erythema (redness) was quantified using a CR300 chromameter (Minolta, Ramsey, N.J.). The difference in erythema (Δa*) between irradiated and non-irradiated skin (a*_{irrad}−a*_{non-irrad}) was calculated for each time point after SSR exposure (Δa*ₜᵢₘₑ). The difference in Δa*time between the investigative and control phases of the study was calculated to determine the effect of the study drug on skin redness after SSR exposure [(Δa*ₜᵢₘₑ−)ᵢₙᵥₑₛₜ−(Δa*ₜᵢₘₑ)_{control}].

Skin thickness, an acute measure of edema, was quantified using a Mitutoyo 9 mm dial caliper (Northamptonshire, UK). Thickness measurements were repeated in triplicate and the mean was used for all calculations. The difference in thickness (Δth) between irradiated and non-irradiated skin (th_{irrad}−th_{non-irrad}) was calculated for each time point after SSR exposure (Δthₜᵢₘₑ). The difference in Δthₜᵢₘₑ between the investigative and control phases of the study was calculated to determine the effect of the study drug on skin thickness after UVR exposure [(Δthₜᵢₘₑ)ᵢₙᵥₑₛₜ−(Δthₜᵢₘₑ)_{control}].

A six millimeter punch biopsy specimen was obtained from the 3MED site 48 hr after irradiation in both the control and investigative phases of the study. RNA was extracted from fresh frozen tissue using the RNeasy Lipid Mini Kit (Qiagen, Redwood City, Calif.), and tissue mRNA expression of TNF-α and iNOS were quantified as previously described. Au et al., J Invest Dermatol., 135(12):2971-81 (2015). A fold change representing the difference in RNA expression between the investigative and control phases of the study was calculated for TNF-α and iNOS [(RNA₄₈ₕᵣ)ᵢₙᵥₑₛₜ/(RNA₄₈ₕᵣ)_{control}].

Unsupervised Hierarchical Clustering of Participants

An exploratory analysis was performed utilizing clusters obtained from the unsupervised hierarchical clustering of individual participants based on similarities in their gene expression profiles, regardless of allocated treatment group. A minimum of three micrograms of total RNA was submitted to the Gene Expression and Genotyping facility at Case Western Reserve University for microarray analysis (Cleveland, Ohio). Human Genome 2.0 ST arrays (Affymetrix, Santa Clara, Calif.) were the chosen platform to analyze transcriptomic changes incurred by treatment. For microarray analyses, normalized linear data post-(Tᵢₙₜₑᵣᵥ) and pre-(T_{control}) study drug intervention were averaged amongst the eighteen participants. The microarray raw data for two participants was not interpretable given poor sample quality. A fold change representing the difference in RNA log expression between the investigative and control phases of the study was determined for each gene for each participant [(RNA₄₈ₕᵣ)ᵢₙᵥₑₛₜ−(RNA₄₈ₕᵣ)_{control})]. Data analysis was performed using Rv3.2.2/Bioconductor (R Studio, Boston, Mass.). The oligo package was used to read, background correct, and normalize the raw sample data using the Robust Multiarray Average algorithm. Bolstad et al., Bioinformatics. 22; 19(2):185-93 (2003). The limma package was used to create a paired design matrix and conduct differential gene expression analysis based on a linear model fit and empirical Bayes methodology. Ritchie et al., Nucleic acids research, 43(7):e47 (2015).

Heatmaps and dendrograms were generated utilizing 26,599 transcripts with unique gene names using GENE-E software. Hierarchical clustering was used to recursively merge samples based on pairwise distance, determined using the 1-Pearson correlation coefficient and average linkage methods. Ingenuity Pathway Analysis (QIAGEN, Redwood City, Calif.) was used to determine statistically significant canonical pathways, predicted up-stream regulators, and biological networks most likely affected by the set of genes differentially expressed for each group of participants.

Tissue arginase-1 mRNA expression was quantified using qRT-PCR as described above. To analyze the expression of the arginase-1 protein in skin, freshly frozen biopsy samples embedded in optimal cutting temperature compound were cut into eight micrometer sections and stained immunofluorescently as previously described (Au et al., supra). CD163 was used as a marker for skin macrophages, and 4′-6-diamidino-2-phenylindole (DAPI) was used to stain nuclear DNA.

Statistical Analysis

Given the pilot nature of the study design, power calculations were not performed. To control for type I error rate, hierarchical closed-testing procedures were utilized for analysis of primary outcomes. Unpaired t-tests were used to compare the means of groups with respect to changes in the primary outcomes at each time point. Fisher's exact test was used for inter-group comparisons of categorical data and for determining significance of the canonical pathways. The Benjamini-Hochberg correction was used to determine differentially expressed genes in the microarray analysis, adjusting for multiple comparisons. Benjamini Y, Hochberg Y., Journal of the Royal Statistical Society Series B (Methodological) 57(1):289-300 (1995). A two-sided p value of 0.05 or less was considered to indicate statistical significance.

All participants completing both phases of the study and receiving the study drug were included in the per-protocol analysis. All statistical comparisons were made between the vitamin D3 treatment groups and the placebo group. Significance of the canonical pathways represents the likelihood that genes in the differentially expressed gene set map to a particular process or pathway more than expected by random chance alone. The Bonferroni correction for multiple comparisons was not utilized given the small overall number of planned comparisons in the primary outcome analyses, as well as the exploratory nature of the post-hoc analyses performed to guide further investigation.

Determination of the Minimal Erythema Dose

The MED is the amount of UVR that will produce minimally perceptible skin erythema a few hours after UVR exposure. MED testing was performed by exposing eight 1 cm² areas on sun-protected buttock skin to increasing doses of UVR using plastic holed templates to ensure that adjacent skin was not irradiated. Total body exposure of a fair-skinned individual to one MED of UVR is approximately equal to the ingestion of vitamin D3 at a dose of 10,000 to 25,000 IU. Holick M F., Lancet, 357(9249):4-6 (2001). Thus, exposure of 1 cm² of skin in this protocol is unlikely to have contributed to an appreciable rise in serum vitamin D3. The duration of irradiation was based on the participants' Fitzpatrick skin type, as previously described. Fitzpatrick T B., Archives of dermatology 124(6):869-71 (1998). Erythema at each site was quantified 24 hr after irradiation using a chromameter (CR300; Minolta, Ramsey, N.J.). The erythema of unexposed skin at each time point served as a baseline. Spectrophotometers measure darkness (L*), hue (b*), and redness (a*) of the skin). Heckman et al., J Vis Exp., 28; (75):e50175 (2013). Larger a* values indicate greater erythema. A linear regression best-fit line was calculated from a plot of the erythema of exposed minus unexposed skin (a*ₑₓₚ−a*ᵤₙₑₓₚ) versus the log of the UVR exposure time. The MED is formally defined as the smallest UVR dose capable of producing a*ₑₓₚ−a*ᵤₙₑₓₚ greater than or equal to 2.5. Solving the linear regression equation for the inverse log of x when Δa* of 2.5 yielded the exposure time in seconds that was required to produce the MED. The ultraviolet B (UVB) irradiance (W/cm²) is the UVB output of a particular SSR devise, and was determined for each participant using a radiometer prior to MED testing. The MED dose (mJ/cm²) of UVB is the product of the irradiance (mJ/sec*cm²) and exposure time (sec). The MED dose of UVB (mJ/cm²) for each participant was calculated using the measured UVB irradiance of the device and the MED exposure time as calculated above.

RNA Expression and Tissue Microarray

RNA (100 ng) was isolated using the Qiagen RNeasy Lipid Tissue Mini Kit (Qiagen Inc., Valencia, Calif.). TNF-α, iNOS, and arginase-1 mRNA expression was quantified using TaqMan Gene Expression Assays and the TaqMan RNA-to-CT 1-Step (Life Technologies, Grand Island, N.Y.), as previously described (Au et al., supra). Gene expression was normalized to the 18s RNA housekeeping gene. Samples were analyzed using a Step-One System (Biosystems, Grand Island, N.Y.) based on the manufacturer's recommendations. For microarray analyses, the gene set of differentially expressed genes was restricted to transcripts with a fold change threshold (Tᵢₙₜₑᵣᵥ/T_{control})≥1.5 or ≤1.5, thereby detecting genes that were reliably and differentially changed by study drug intervention.

Immunofluorescence Staining

Primary antibodies, including mouse monoclonal CD163 (EDHu-1, Bio-Rad, Hercules, Calif.), and rabbit polyclonal arginase-1 (Novus Biologicals, Littleton, Colo.) were diluted 1:50 and 1:100 respectively in 10% goat serum in phosphate buffered saline. Arginase-1 was incubated for 1 hr at room temperature, and CD163 was incubated overnight at 4 degrees Celsius. Secondary antibodies, including goat anti-mouse (Alexa Fluor 488) and goat anti-rabbit (Alexa Fluor 647) (Thermo Fisher, Grand Island, N.Y.), were diluted 1:2000 in phosphate buffered saline. Eight micrometer tissue sections were imaged using a Leica DMI 6000 B inverted microscopy (Leica Microsystems, Wetzlar, Germany) with a Retiga Aqua blue camera (Q-Imaging, Vancouver, British Columbia), and subsequently analyzed using Metamorph Imaging Software (Molecular Devices, Downinton, Pa.).

### Example 2: Oral Vitamin D Attenuates Inflammation from Nitrogen Mustard

Two adults 18 years and older were screened for eligibility. The subjects were tested with topical application of nitrogen mustard in the FDA-approved form called Valchlor™ (0.016% mechlorethamine gel) for 1 hour. Results demonstrated no erythema or swelling and no altered skin sensation were observed in the study subjects over the period of 1 week. Skin biopsies at 48 hrs from each subject showed no changes on histopathology or increase in pro-inflammatory mediators by qRT-PCR. In view of these findings, a modified protocol with dose escalation exposure time and concentration of topical nitrogen mustard was developed. Six adults were then screened for eligibility and 4 were enrolled based on the new protocol of topical Valchlor exposure for 48 hours. Reaction to Valchlor™ was confirmed before the subjects were allowed to proceed with the placebo vs. Vitamin D3 intervention phase of the study.

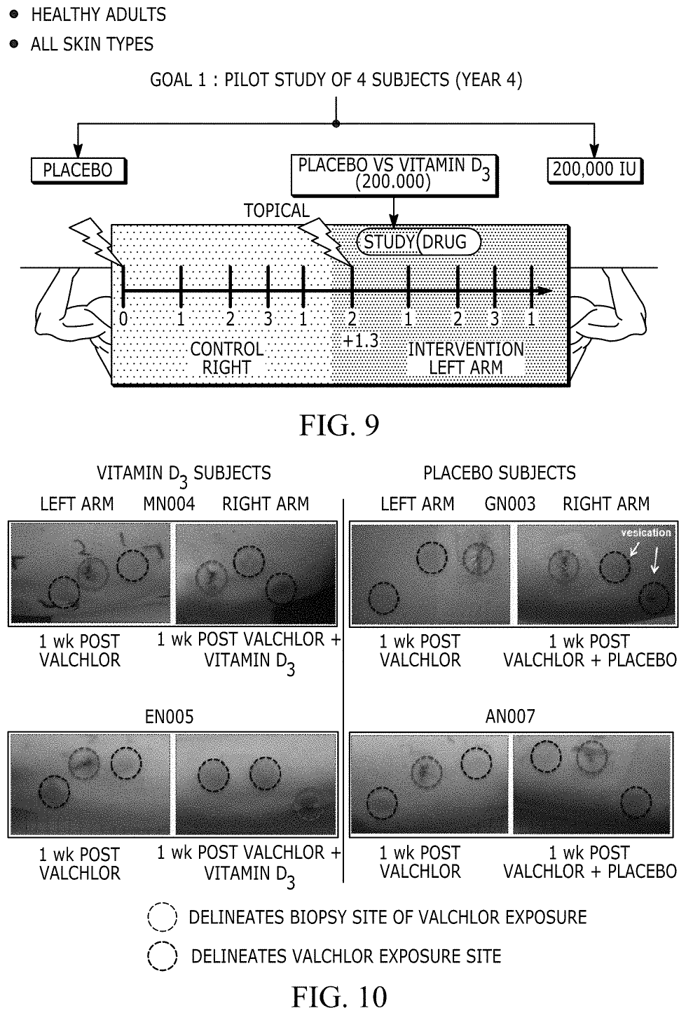

Similar to the study described in Example 1, the Valchlor™ trial was designed in a parallel fashion with exposure to topical Valchlor™ occurring on a single arm without study drug administration (control phase), followed by exposure to Valchlor™ on the contralateral arm two weeks later with study drug administration (investigative phase) (FIG. 9). A dose of 200,000 I.U. vitamin D3 is effective. To maintain rigor and remove bias, a placebo control group is used. All studies conducted were double-blinded.

Four subjects were enrolled. For all subjects, exposure to Valchlor™ was performed to each arm in triplicates for 48 hours. At 72 hours, one of the sites is biopsied for histological studies and quantitation of pro-inflammatory factors by qRT-PCR. Below is the gross observation of all subjects (arm 1 and arm 2 at 1 week post Valchlor™ exposure) (FIG. 10).

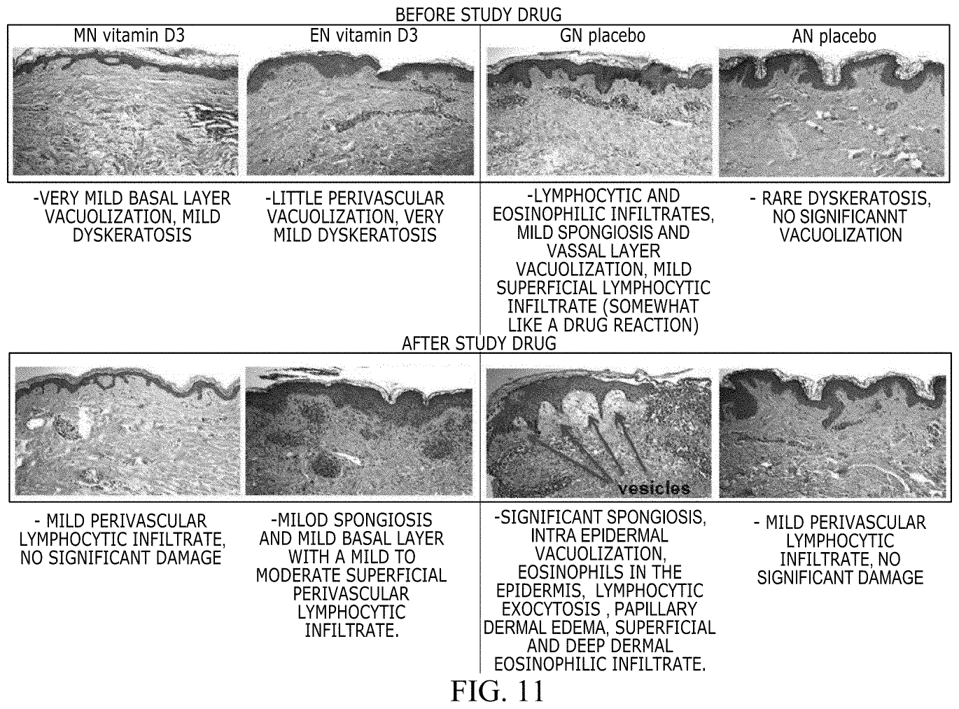

The gross observations correlate with the histopathology. In all subjects, arm 1 demonstrates basilar vacuolization and dyskeratosis of the keratinocytes at the dermal-epidermal junction following exposure to Valchlor™ (FIG. 11). There is also presence of a superficial lymphohistiocytic inflammation which explains the gross observation of skin redness. Further analysis of the preliminary data reveal that exposure to Valchlor™ on arm 1 (control phase) may potentiate a more rigorous reaction when a subject is then exposed to Valchlor on arm 2 two weeks later. In all 4 subjects, the baseline skin erythema was higher in arm 2 than baseline arm 1. This may represent a “sensitization and challenge” reaction. This is supported with a slight increase in inflammatory infiltrates in the skin on histopathology. However, in 1 study subject that received placebo, the potentiated reaction to Valchlor™ resulted in vesicle formation (subject GN003) which is also supported by histology. There is no vesication formation in both study subjects receiving vitamin D3. Our goal was to demonstrate a 25% reduction in vesication by histology. Based on our small sample size for both treatment groups (n=2), there was 50% vesication with placebo and 0% vesication with vitamin D3 intervention.

In the study of the first 4 subjects (2 placebo and 2 vitamin D3), oral vitamin D3 intervention resulted in a reduction in erythema following Valchlor™ exposure over the 6-day period (slope calculated through linear regression). The values are calculated based on the slope (arm 2-arm 1 redness) over several time points. See Table 3. However, the 2 subjects that received placebo demonstrated an increase in erythema over the 6 day period (slope value of +0.359). In contrast, the 2 subjects that received vitamin D3 had a reduction in erythema over the same period (slope value −0.491). The goal was to demonstrate a 25% reduction in erythema in 2 subjects treated with vitamin D3 compared to control. The preliminary data here demonstrate a drastic reduction in erythema.

TABLE 3
Net Erythema Reduction
NET ERYTHEMA REDUCTION

| slope = linear | | | average slope | |
|---|---|---|---|---|
| regression calculated | | slope | n = 2 | |
| from day 0 | | arm 2 minus | arm 2 minus | |
| through day 6 | | arm 1 | arm 1 | |
| Placebo | AN007 | 0.215 | 0.35945 | positive change in |
| | GN003 | 0.5039 | | slope indicates |
| | | | | arm 2 to be |
| | | | | higher or equal |
| | | | | to arm 1 |
| D3 | MN004 | −0.1918 | −0.49185 | negative change |
| | EN005 | −0.7919 | | in slope indicates |
| | | | | arm 2 slope to be |
| | | | | lower than arm 1 |

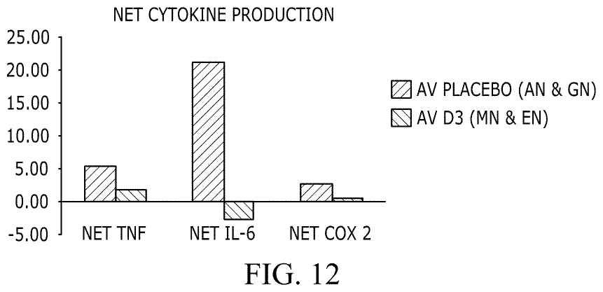

Another goal was to demonstrate reduction in pro-inflammatory factors in the skin. We observed that oral vitamin D3 (n=2) resulted in reduction of three key pro-inflammatory factors following NM exposure from skin biopsies: 66% reduction in TNF-α; 112% reduction in IL-6, and 80% reduction in COX-2. No demonstrable increase in iNOS was observed at baseline to make comparison with intervention with vitamin D (FIG. 12).

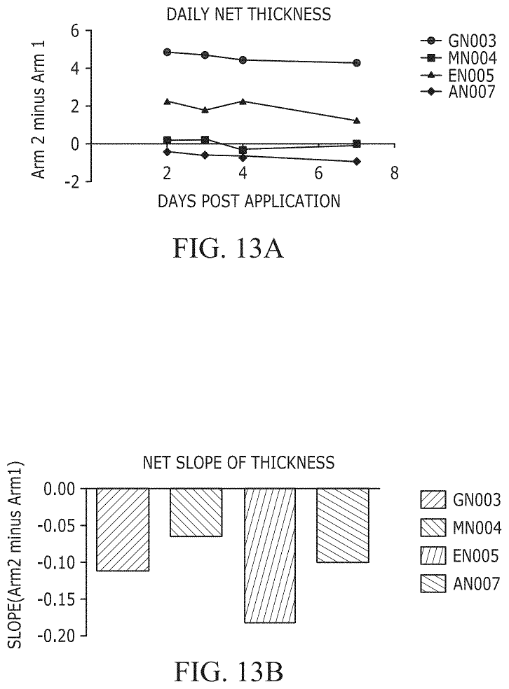

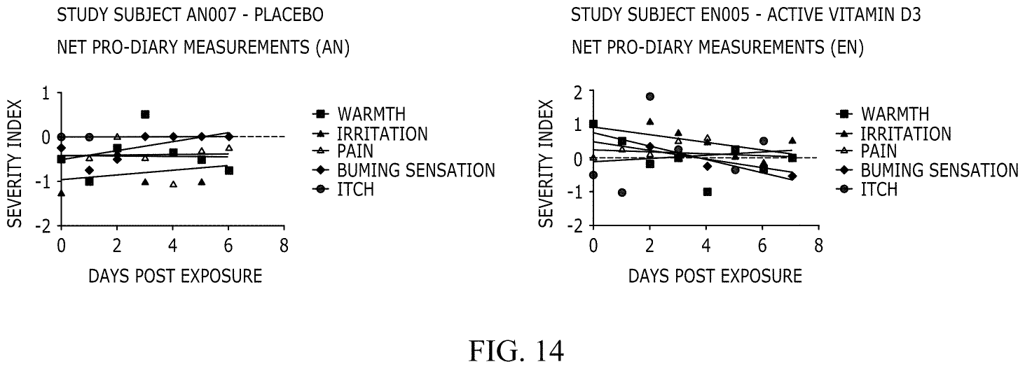

In all 4 subjects, there was a trend towards reduction in skin thickness measured over 7 days. A priori, the prediction would be that subjects receiving placebo would have a no change or increase in skin thickness (arm 2-arm 1). Consistent with our hypothesis, the two subjects receiving vitamin D3 intervention displayed an increase, albeit modest, reduction in skin thickness (subjects MN004 and EN005) compared to placebo (FIGS. 13A and 13B).

For this study, we also added a component of “subject self-reported skin symptoms” using a Pro-Diary™ wristwatch. The programmed device prompted subjects to answer questions, rating skin sensation severity (scale 0-9) several times a day over the entire week that they are tested with Valchlor™. Subjects were blinded to their treatment allocation. The results from 2 subjects (1 in each treatment group) show that patient reported symptoms were decreased with vitamin D3 intervention. Results were obtained from only 2 subjects as the other 2 subjects had compliance issues with wearing the device and failure to input data (FIG. 14 and Table 4). Additional skin symptoms were recorded including skin pain, burning sensation, and itch. In the current preliminary data, no observable difference was observed with n=1 for each group.

TABLE 4
Net Skin Symptoms Measurement

| | Net Skin Symptoms Measurements | | | | | | | |
|---|---|---|---|---|---|---|---|---|
| | Day 0 | Day 1 | Day 2 | Day 3 | Day 4 | Day 5 | Day 6 | Slope |
| | Warmth | | | | | | | |
| AN007 (Placebo) | −0.5 | −1.0 | −0.3 | 0.5 | −0.3 | −0.5 | −0.8 | 0.006071 ± 0.9 |
| EN005 (D3) | 1.0 | 0.5 | −0.2 | 0.0 | −1.0 | 0.3 | −0.3 | −0.1296 + 0.083 |
| | Irritation | | | | | | | |
| AN007 (Placebo) | −1.3 | −1.0 | −0.3 | −1.0 | −0.3 | −1.0 | −0.8 | 0.05357 ± 0.07 |
| EN005 (D3) | 1.0 | 0.5 | 1.1 | 0.8 | 0.5 | 0.1 | −0.2 | −0.1202 ± 0.05 |

### Example 3: Autophagy Reprogramming by Vitamin D Promotes Suppression of UV-Induced Inflammation Via Macrophage Polarization

Exposure to ultra violet radiation (UVR) inflicts acute damage to the skin to initiate a cascade of inflammatory reactions that exacerbate tissue destruction resulting in delayed wound repair. The damaged epidermis generates free radicals thus exaggerating skin inflammatory response through massive infiltration of immune cells including neutrophils, DC and macrophages. So far, a myriad topical emollients and steroids have been the mainstay of current therapy for treating skin inflammation however such approaches have met with temporary success with some cases becoming increasingly refractory to steroid application with repeated use. In a murine model of chemical-induced cutaneous injury we have previously demonstrated that administration of 25(OH)D, vitamin D (VitD) IP, 1 h following nitrogen mustard exposure, was sufficient to prevent acute skin and systemic inflammation and rescue mice from extensive tissue damage and mortality (JID 2015). To further establish the efficacy of VitD as a skin anti-inflammatory agent, we have recently demonstrated in a human translational study that a single high dose of oral cholecalciferol (VitD) diminished acute skin inflammation induced by UV radiation to accelerate skin recovery possibly in an arginase1 dependent manner. Both studies indicate that VitD mediated attenuation of cutaneous inflammation is accompanied by the preferential expansion of anti-inflammatory macrophages. This is consistent with other studies showing that recovery of skin injury is mediated chiefly by anti-inflammatory (M2) macrophages that suppress inflammation and augment epidermal regeneration.

Macrophages, being one of the early responders of tissue inflammation, generate reactive species with secretion of cytokines and chemokines that further exacerbates the inflammatory milieu. Suppression of inflammation is mediated chiefly by the differentiation and stabilization of anti-inflammatory M2 macrophages however the exact cues that prompts the M2 macrophages to quell inflammation at the inflamed site is unknown. Our current study explores the mechanism by which vitamin D (25(OH)D (VitD) modulates early events of acute inflammation in a mouse model of UV induced skin inflammation.

Of the myriad genes that VitD is known to influence, emerging data indicate that VitD may confer its immunoregulatory effects through modulation of autophagy, a cellular degradative pathway that clears internalized damaged proteins to maintain homeostasis. Several studies implicate a health promoting role for autophagy through tumor suppression, antimicrobial defense, cellular longevity, inhibition of neurodegenerative diseases, cardiac hypertrophy and atherosclerosis and increased sensitivity to insulin. In all these pathologies autophagy plays a pleiotropic role to promote diverse outcomes ranging from cell death or cell survival depending on the molecular signals in its microenvironment. Emerging evidence demonstrates an immunomodulatory role of autophagy in the skin to counter environmental stressors through inactivation of the inflammaasome. Impaired autophagy in keratinocytes triggered skin autoinflammation exacerbated by uncontrolled cytokine production. Animal models of infection and chronic inflammation implicate a role of autophagy associated with a disbalance of M1 and M2 macrophages. These data identify a pivotal role of autophagy in the induction and regulation of inflammatory responses in the skin. However, the link between autophagy and VitD mediated immunomodulation remains to be elucidated.

Here we investigated the contribution of macrophage autophagy in VitD protection of skin inflammation following UV radiation. VitD mediated attenuation of skin inflammation was consistently associated with enhanced UV-induced autophagy in the skin specifically in the CD206+ M2 macrophages populating the inflammation bed with significant protection from UV-induced apoptosis. Inhibition of autophagy curbed LC3 expression over time while drastically impairing VitD mediated protection by increasing cytokine production, depleting M2 macrophages and causing massive epidermal apoptosis. Take together we propose that VD induction of autophagy is a potential therapeutic option for treating UV-induced acute cutaneous inflammation via expansion of functional anti-inflammatory macrophages expressing arginase1.

### Results

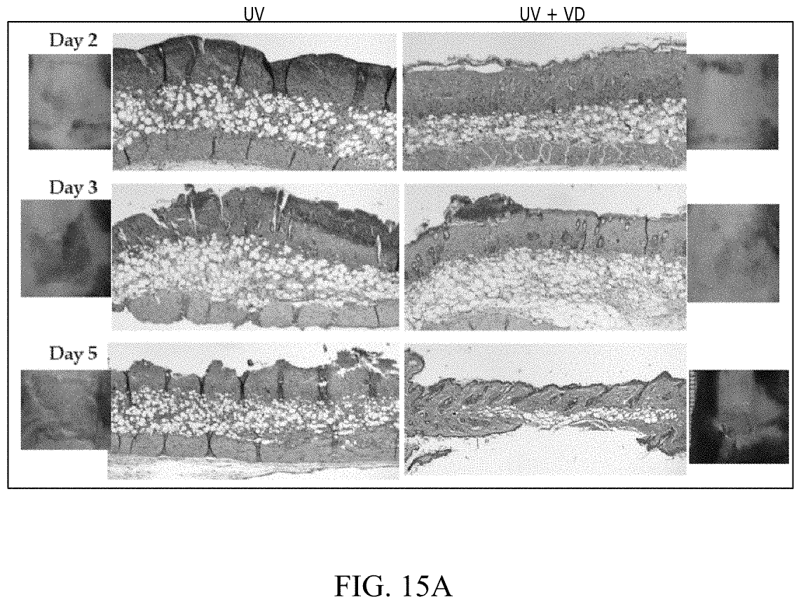

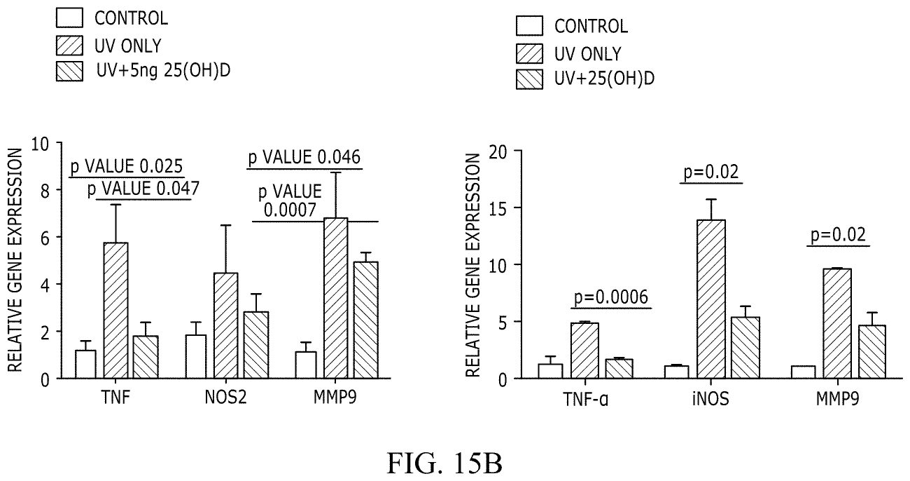

25(OH)D attenuates skin inflammation in a mouse model of UV skin exposure. One of the remarkable pleiotropic effects of vitamin D is immune suppression which has consequently been employed to modulate inflammation. To determine 25(OH)D immunosuppressive properties in the skin following inflammation from UV radiation, mice were irradiated with an erythemogenic dose of UV radiation (100 mJ/cm²) that is shown to cause epidermal damage and dermal inflammation. At 48h post UV exposure and thereafter, gross images of mouse back displayed redness and inflammation that correlated with destruction of dermal and epidermal architecture. Days 3 and 5 without treatment made the skin wounds progressively worse with complete erosion of the epidermis, edema and disruption of the adipocyte layer. Intervention with a single injection IP of 25(OH)D, 1 hour after exposure, delayed skin inflammation, arrested skin wound progression and accelerated wound repair (day 5). Histopathological examination of skin sections revealed that VitD treatment was associated with protection of histological architecture in dermis and epidermis. Assessment of molecular markers of inflammation before and after treatment with the study drug indicate that, gene expression of iNOS, TNF and MMP9 levels were significantly suppressed at 48 h post UV and remained reduced at 72 h (FIG. 15).

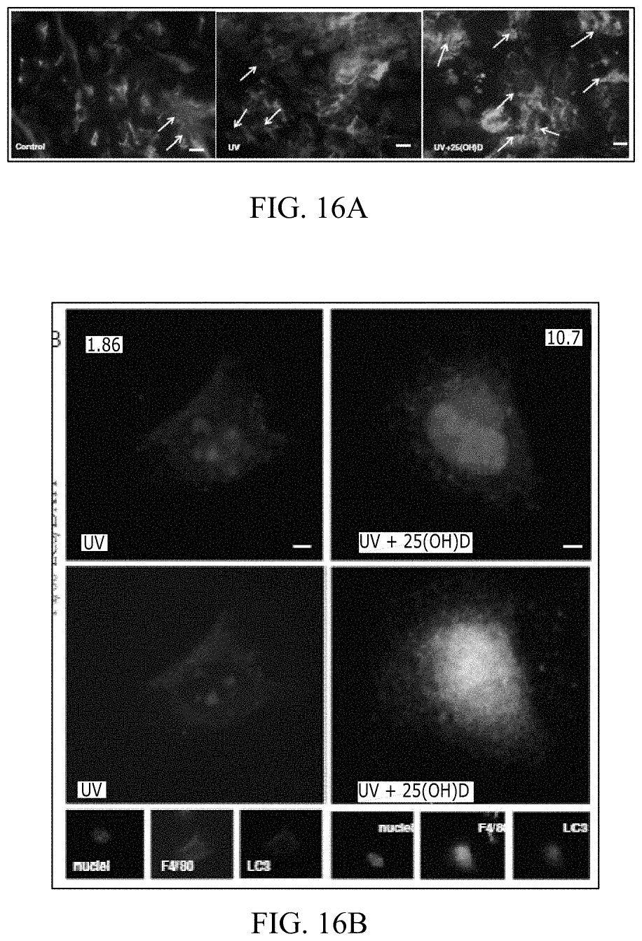

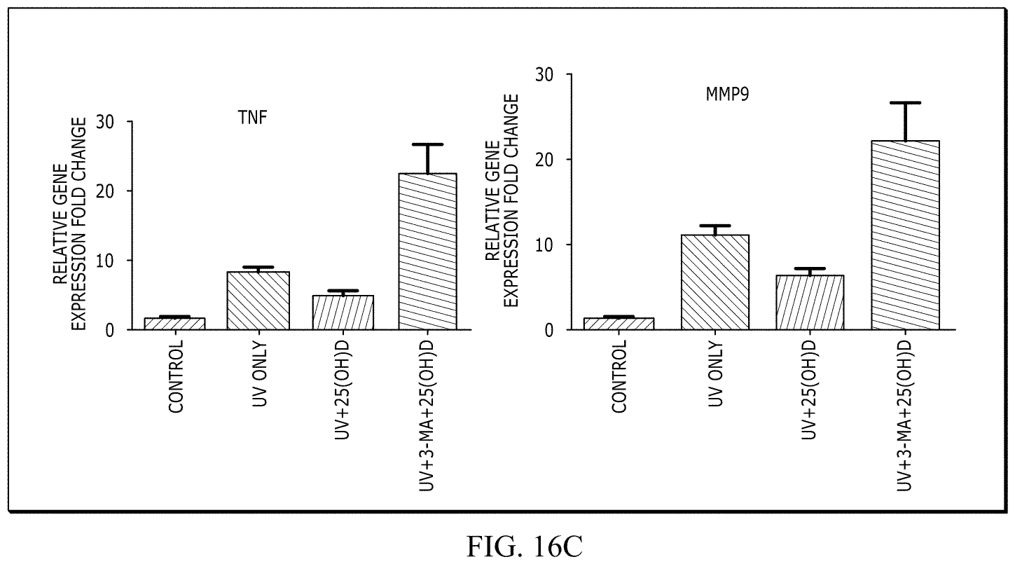

25(OH)D enhances macrophage-specific autophagy. Given that VD induces autophagy in other models with expansion of anti-inflammatory macrophages, we sought to localize macrophage specific autophagy induced by VD in skin tissue of mice 48 h following UV exposure. While UV induced autophagy in macrophages, a number of F480− cells were positive for autophagy. Interestingly treatment with VD detected greater abundance of LC3 co-localizing with F480+ macrophages (FIG. 16A). Classically autophagy is detected by the presence of discrete punctae that distribute throughout the cell designating autophagolysosomes. To ascertain that autophagic punctae were detected in our model, cells from UV exposed cutaneous tissue were stained and imaged to reveal that compared to UV induced autophagy, treatment with VD elevated macrophage-specific autophagy (LC3 punctae) by 5-fold (FIG. 16B).

Inhibition of autophagy in vivo using 3-methyladenine not only inhibited UV induced autophagy and VD induced autophagy but reversed VD mediated protection from inflammation (FIG. 16C).

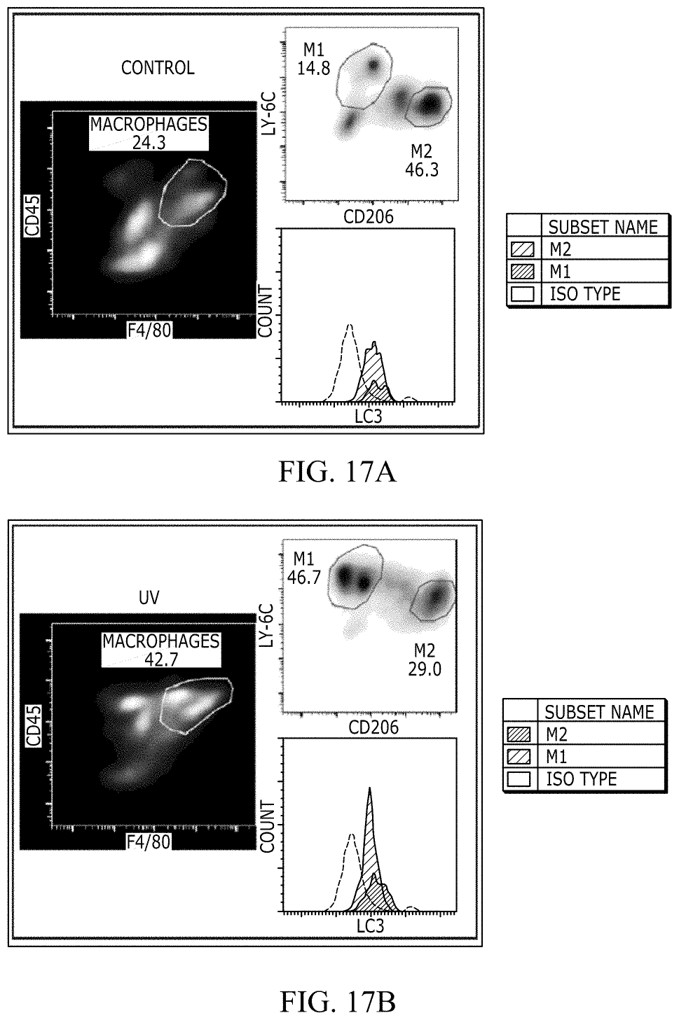

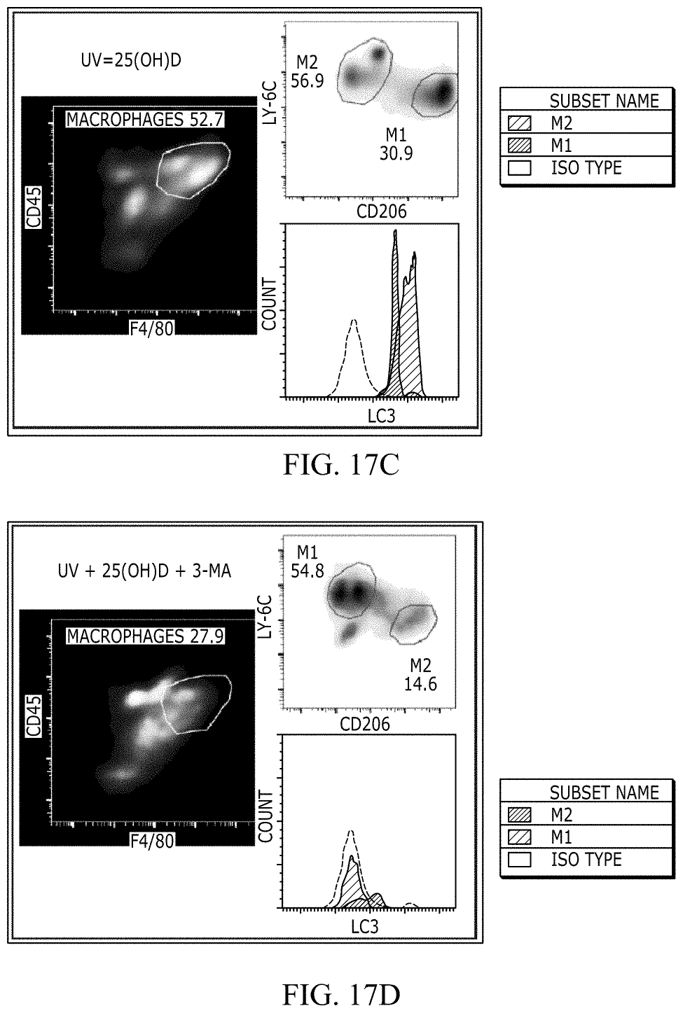

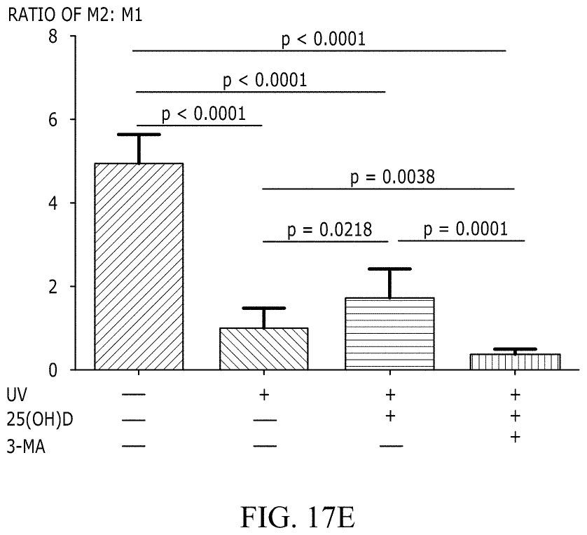

25(OH)D repopulates CD206+ macrophages in an autophagy dependent manner. Since VD suppresses inflammation through expansion of anti-inflammatory M2 macrophages, we next characterized the cells infiltrating the UV skin wound. Analyses by flow cytometry distributed the myeloid cells into 2 distinct populations—CD45+F480+Ly6C+CD206− which were the classic M1 macrophages and the other—CD45+F480+Ly6C−CD206+ cells or M2 macrophages. Representative dot plots demonstrate that control mice harbor an abundance of M2 cells (3-fold more M2 than M1) in the skin possibly to maintain quiescence and homeostasis. Irradiated mice show a complete disappearance of M2 cells with increase of M1 cells that are LC3+. In contrast VD treated mice exhibit more M2 cells co-localizing with LC3. Lastly inhibition of autophagy in presence or absence of VD reduced M2 cells suggesting that VD expanded M2 in an autophagy dependent manner (FIG. 17A-E). Thus 25(OH)D mediated immunosuppressive effects is regulated by the relative abundance of M2 to M1 cells.

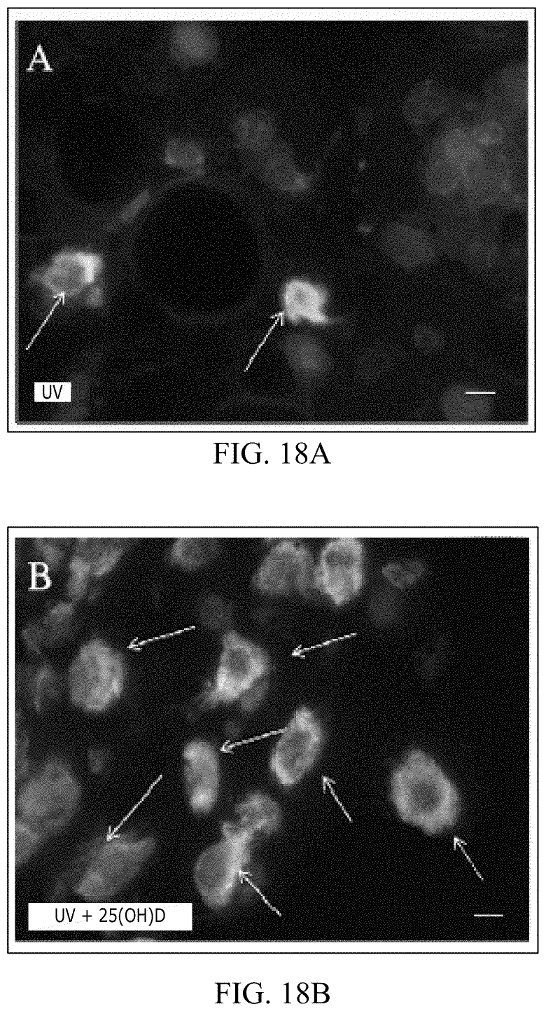

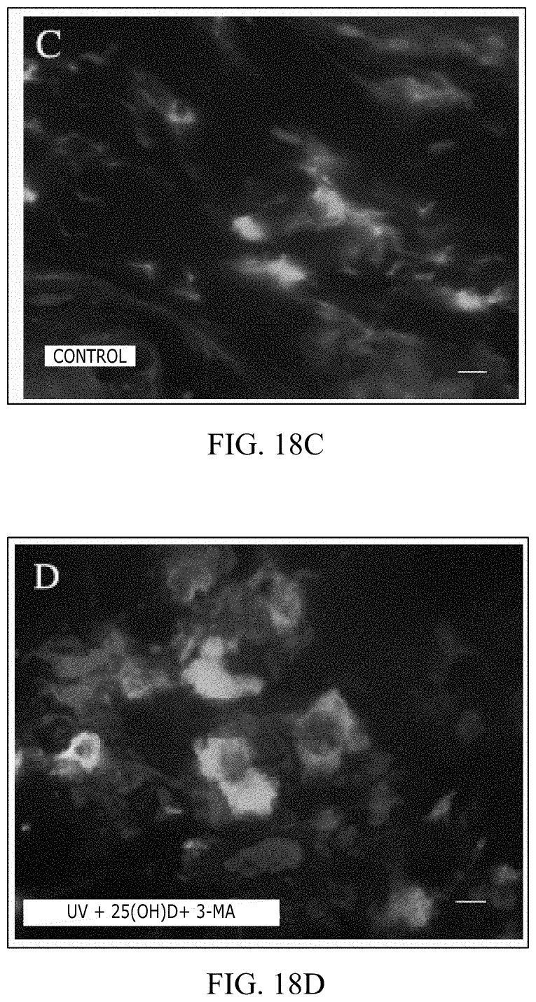

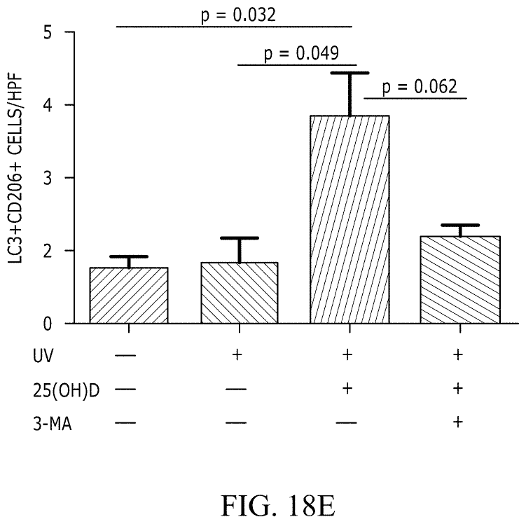

Autophagy is enhanced selectively on CD206-expressing macrophages. To further validate that VD enhances autophagy specifically in anti-inflammatory macrophages, UV irradiated mouse skin tissue before and after treatment was stained to detect and co-localize LC3 with CD206. VD treated mice exhibited a significantly increased number of cells in the dermis that were LC3+CD206+. Inhibition of autophagy by 3MA abolished LC3 expression but still exhibited CD206+ cells suggesting that these cells may be less functional than their VD counterpart (FIG. 18).

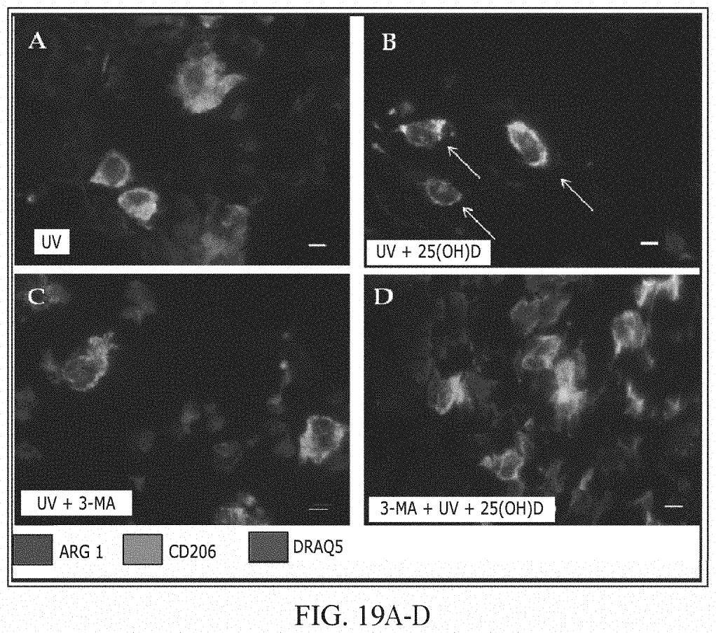

Functionally competent M2 macrophages confer protection against UV mediated inflammation. Since Arg1 is not only an M2 marker but enhanced expression of arg1 allows for deviating the cells away from generating NO from iNOS activity. We show that mice treated with VD selectively stain for arg1 in CD206+ cells whereas inhibiting autophagy with 3MA diminished arg1 expression in these cells (FIG. 19).

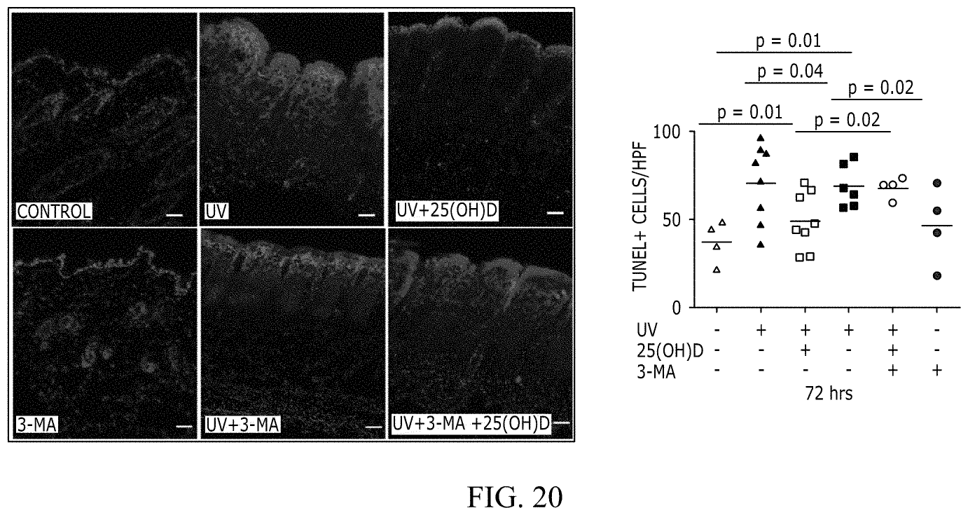

VitD protects mice from UV induced apoptosis in an autophagy dependent manner. To assess the extent of skin tissue damage, parallel studies sought to determine cellular apoptosis caused by UV. 25(OH)D treatment significantly diminished UV mediated apoptotic cell death compared to massive cellular apoptosis observed in untreated UV irradiated mice. Massive epidermal and dermal tissue destruction and cellular apoptosis was observed in UV-irradiated mice treated with 3-MA compared to other treatment conditions. Cellular apoptosis is quantified as TUNEL+ cells (FIG. 20). These data introduce a new paradigm in immune response in skin injury. The ability of vitamin D to enhance cell “fitness” by upregulation of autophagy represents potential new avenues that can be translated for clinical use in skin repair.

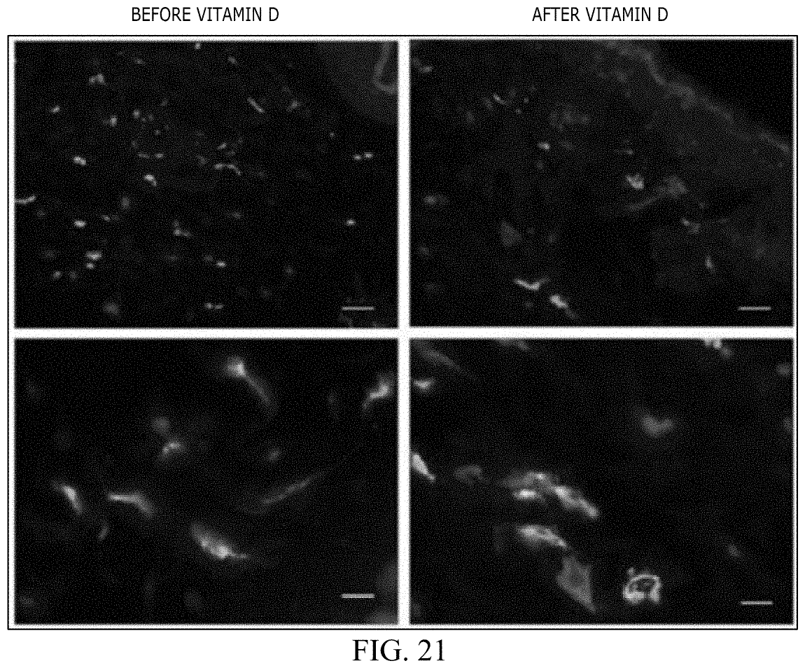

VitD induced autophagy is confirmed in human skin. To determine that our findings in the animal model has human and translational relevance, we stained human skin biopsies for LC3 and macrophage marker CD163. The skin biopsies were obtained from a double-blinded placebo controlled study of healthy subjects exposed to UV followed by treatment with oral vitamin D. The results show that vitamin D treatment indeed upregulates cells that co-localize LC and CD163 (FIG. 21).

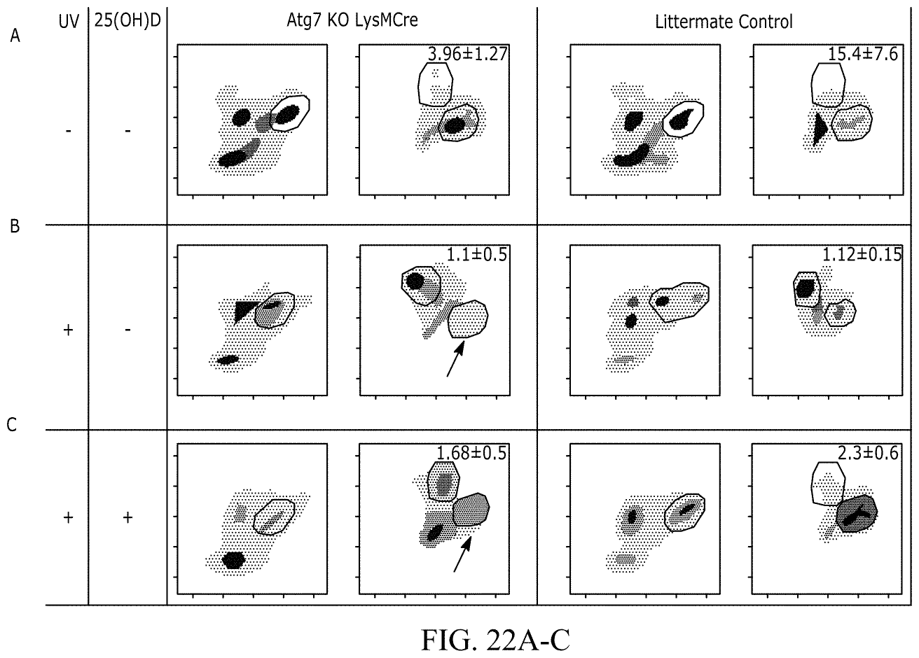

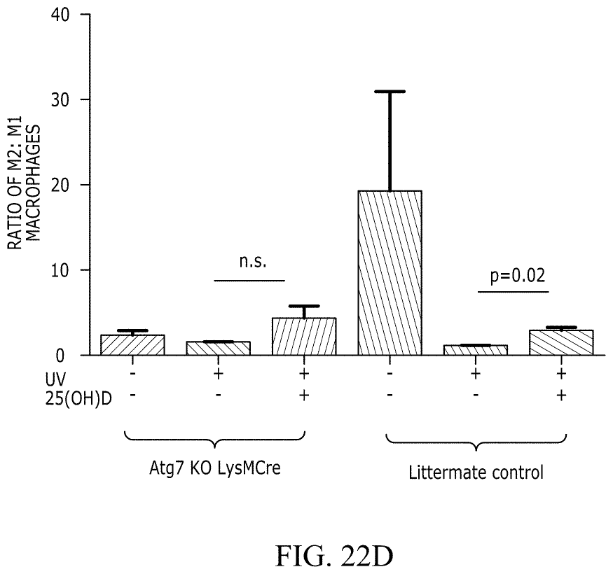

Depletion of autophagy specifically in myeloid cells depletes M2 macrophages, reduces M2:M1 ratio and impairs VitD ability to attenuate inflammation. Lastly, we wanted to confirm our findings that inhibition of autophagy in macrophages results in altered beneficial macrophage ratios in the skin and that this is an important mechanism by which vitamin D mediates it effect. We performed breeding experiments to generate the conditional knockouts using the Cre-LoxP technology of the Atg7-LoxP gene crossed with LysM-Cre mice (Jackson Laboratories). The resulting animals are selectively deficient in the autophagy gene in macrophages. The results show that autophagy is required for vitamin D mediated increase in M2:M1 ratios (FIG. 22A-D).

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of treating or preventing skin damage in a subject in need thereof, comprising administering to the subject a therapeutically effective amount of an autophagy activator.

2. The method of claim 1, wherein the autophagy activator is vitamin D, a vitamin D analog, or a vitamin D metabolite.

3. The method of claim 2, wherein at least 50,000 IUs of the vitamin D, vitamin D analog, or vitamin D metabolite are administered to the subject.

4. The method of claim 2, wherein at least 100,000 IUs of the vitamin D, vitamin D analog, or vitamin D metabolite are administered to the subject.

5. The method of claim 2, wherein at least 200,000 IUs of the vitamin D, vitamin D analog, or vitamin D metabolite are administered to the subject.

6. The method of claim 1, wherein the autophagy activator is vitamin D.

7. The method of claim 1, wherein the autophagy activator is an mTOR pathway inhibitor.

8. The method of claim 1, wherein the skin damage is caused by one or more of a chemical burn (including but not limited to as caused by vesicants), a radiation burn (including but not limited as caused by UV and X-rays), a heat burn (including but not limited as caused by fire, steam, hot objects, and hot liquids), a cold burn, an electrical burn, a friction burn, or trauma.

9. The method of claim 8, wherein the vesicant is one of a weaponized vesicant (including but not limited mustard gas, nitrogen mustard, and sulfur mustard) or a chemotherapeutic vesicant (including but not limited to mechlorethamine and doxorubicin).

10. The method of claim 1, wherein the skin damage is skin burn injury.

11. The method of claim 1, wherein the skin damage is a result of radiation exposure.

12. The method of claim 11, wherein the radiation is sunlight.

13. The method of claim 1, wherein the subject is human.

14. The method of claim 1, wherein the autophagy activator is administered orally.

15. The method of claim 1, wherein the method includes treating skin damage in a subject.

16. The method of claim 1, wherein the method includes preventing skin damage in a subject.

17. The method of claim 1, wherein the autophagy activator is administered as part of a pharmaceutical composition.

18. A method of monitoring the immunomodulatory effects of an autophagy activator on skin damage in a subject, the method comprising administering the authophagy activator to the subject and determining the levels of arginase-1 in a biological sample obtained from the subject, wherein an increased level of arginine-1 indicates the autophagy activator is having an effective immunodulatory effect.

19. The method of claim 17, wherein the autophagy activator is vitamin D.

20. A method for correlating the dosage of an oral administration of cholecalciferol in humans with the dosage of an intraperitoneal injection of 25(OH)D in mice.
